(19) United States
(12) Patent Application Publication
(10) Pub. No.: US 2018/0193361 A1
(43) Pub. Date: Jul. 12, 2018

(54) GUT ANTI-INFLAMMATORY AGENTS FOR REGULATION OF HIGH BLOOD GLUCOSE LEVELS

(71) Applicant: UNIVERSITY HEALTH NETWORK, Toronto (CA)

(72) Inventors: Daniel Aaron WINER, Thornhill (CA); Shawn Michael WINER, Thornhill (CA); Sue Yu-Sue TSAI, Toronto (CA); Helen LUCK, Toronto (CA)

(21) Appl. No.: 15/563,392

(22) PCT Filed: Apr. 1, 2016
(86) PCT No.: PCT/CA2016/000101

§ 371 Date: Sep. 29, 2017

Related U.S. Application Data
(60) Provisional application No. 62/142,007, filed on Apr. 2, 2015.

(51) Int. Cl. A61K 31/606; A61P 3/10; A61K 31/635; A61K 31/196

(52) U.S. Cl. CPC A61K 31/606; A61K 31/196; A61K 31/635; A61P 3/10

(57) ABSTRACT

A method of treating high blood glucose levels is disclosed. The method includes administering gut anti-inflammatory agents such as mesalamine (5-aminosalicylic acid), sulfasalazine, asacol, delzicol, pentasa, lialda, apriso, olsalazine, balsalazide and GED-0507-34, or pharmaceutically acceptable salts, solvates, or esters of any of the foregoing

### CROSS-REFERENCE TO RELATED APPLICATIONS

This application claims the benefit of U.S. Provisional Patent Application Ser. No. 62/142,007, filed Apr. 2, 2015, which is hereby incorporated by reference in its entirety.

### FIELD

The present disclosure relates generally to gut anti-inflammatory agents and methods of using same for regulation of glucose levels and in particular high blood glucose levels.

### BACKGROUND

Obesity and its associated metabolic abnormalities including Type 1 diabetes, Type 2 diabetes (T2D) and the precursors, insulin resistance (IR), and/or high glucose levels have become global diseases that carry considerable morbidity and mortality (Johnson and Olefsky, 2013). Obesity-related IR can arise through multiple pathways, but chronic inflammation in visceral adipose tissue (VAT) has become a prominent pathological mechanism (Gregor and Hotamisligil, 2011; Odegaard and Chawla, 2013). Cells of both the innate and adaptive immune system residing in VAT have been shown to play a key role in IR. More specifically, M1 macrophages, interferon (IFN)γ-secreting Th1 T cells, CD8+ T cells, and B cells promote IR, in part, through secretion of pro-inflammatory cytokines (Lumeng et al., 2007; Nishimura et al., 2009; Winer et al., 2011; Winer et al., 2009a). In contrast, Foxp3+ regulatory T cells (Tregs), eosinophils, Th2 T cells and type 2 innate lymphoid cells (ILC2) are associated with protection from IR through local control of VAT inflammation (Feuerer et al., 2009; Molofsky et al., 2013; Winer et al., 2009a; Wu et al., 2011).

In addition to VAT, recent evidence has pointed to the bowel as a key site that becomes altered in obesity-related IR (Johnson and Olefsky, 2013). Obesity and its metabolic abnormalities have been associated with alterations in the composition of the gastrointestinal flora, known as dysbiosis, which can impact body fat, systemic inflammation and IR (Backhed et al., 2004; Backhed et al., 2007; Membrez et al., 2008; Turnbaugh et al., 2006). Under normal physiological conditions, dysbiosis is kept in check through maintenance of an intact intestinal barrier, characterized by increased mucus, transforming growth factor (TGF)-β, interleukin (IL)-10, IL-22, and luminal secretion of IgA (Brown et al., 2013).

Dysbiosis is believed to cause low-grade inflammation both systemically, through enhanced leakage of bacterial products such as lipopolysaccharides (LPS), and locally in the small bowel and colon (Cani et al., 2007; de La Serre et al., 2010). Systemically, some of these bacterial products, including intestinal-derived antigens, are also thought to accumulate in VAT and potentiate inflammation in this metabolic tissue (Caesar et al., 2012; Wang et al., 2010). Thus, manipulation of the gut barrier to reduce leakage of LPS, through use of cytokines like IL-22, has been associated with improved insulin sensitivity (Wang et al., 2014). Locally, in the bowel, increased tumor necrosis factor alpha (TNFα) and NF-κB activation have been demonstrated in the ileum, while IL-1β and IL-12p40 levels are elevated in colons of HFD-fed mice (Ding et al., 2010; Li et al., 2008). However, data are lacking on the local effects of HFD on most immune cell populations in the gut, as well as their function in IR.

In IR and T2D, treatment with systemic anti-inflammatory therapies such as salicylates and IL-1β antagonists has shown some efficacy in clinical trials (Goldfine et al., 2010; Larsen et al., 2007), and systemic targeting of T and B cells has shown positive effects in rodent models (Winer et al., 2011; Winer et al., 2009a). However, many systemic immune modulators carry potential serious side effects; thus, the development of locally active, well-tolerated and efficient therapies is a principal goal of IR therapy research.

It is, therefore, desirable to provide one or more compounds that can be useful in regulating glucose levels, particularly high glucose levels resulting from glucose intolerance and/or insulin resistance, which may be obesity related, or related to type 1 or type 2 diabetes, with an improved activity profile as compared with the prior art.

### SUMMARY

It is an aspect to provide a method of treating high blood glucose, by selecting a patient having high blood glucose levels and administering gut anti-inflammatory agent to the patient.

In one embodiment, the high blood glucose is as a result of insulin resistance, glucose intolerance, type 2 diabetes or obesity. In another embodiment the insulin resistance is as a result of obesity. In another aspect the glucose intolerance is as a result of type 1 diabetes, type 2 diabetes or obesity.

In another embodiment, the patient is selected on the basis of demonstrating insulin resistance. In one aspect the insulin resistance is as a result of the patient being obese.

In another embodiment, the patient is selected on the basis of demonstrating glucose intolerance. In one aspect the glucose intolerance is as a result of the patient having type 1 diabetes, type 2 diabetes or being obese.

In another aspect the patient is selected as having high blood glucose on the basis of the results of a fasting plasma glucose test, and oral glucose tolerance test, a random plasma glucose estimate, or an A1C test.

In yet another aspect, a method of treating high blood glucose is provided, where the method encompasses selecting a patient having obesity, type 1 diabetes or type 2 diabetes and administering gut anti-inflammatory agent to the patient.

In one embodiment the gut anti-inflammatory agent is a PPAR gamma analogue, or a pharmaceutically acceptable salt, solvate, or ester of the PPAR gamma analogue.

In a further aspect the gut anti-inflammatory agent is mesalamine (5-aminosalisylic acid, 5-ASA) or a derivative, analogue, prodrug or a pharmaceutically acceptable salt, solvate, or ester of any of the foregoing.

In a further aspect the gut anti-inflammatory agent is mesalamine, sulfasalazine, asacol, delzicol, pentasa, lialda, apriso, olsalazine, balsalazide, or GED-0507-34 or a pharmaceutically acceptable salt, solvate, or ester of any of the foregoing and the PPAR gamma analogue is balsalazide or GED-0507-34.

In a further aspect the route of administration is one of orally, intravenously, intraperitoneally, and rectally.

Other aspects and features of the present disclosure will become apparent to those ordinarily skilled in the art upon review of the following description of specific embodiments in conjunction with the accompanying Figures.

### BRIEF DESCRIPTION OF THE DRAWINGS

Embodiments of the present disclosure will now be described, by way of example only, with reference to the attached Figures.

FIG. 1 in one embodiment shows HFD associated with a pro-inflammatory shift in intestinal immune cells. (A) Intracellular cytokine staining in T cells from colon or (B) small bowel lamina propria after 12-16 weeks of HFD (*P=0.005 for Th1, *P=0.02 for CD8, *P=0.02 for Tregs, n=3-4 experiments, 8-10 mice, for colon; *P=0.03 for Th1, *P=0.046 for CD8, *P=0.002 for Tregs, n=5-6 experiments, 10-12 mice, for small bowel). (C) Intracellular cytokine staining in γδ T cells from colon or (D) small bowel lamina propria after 12-16 weeks of HFD feeding (*P=0.02 for colon, *P=0.001 for small bowel, n=4 experiments, 10 mice, for colon and n=4-5 experiments, 10 mice, for small bowel). (E) T-bet staining (far left), Foxp3 (middle), and CD8+ (far right) in colon and ileum of human subjects with lean or obese BMI (*P=0.04, n=7 for colon, *P=0.02, n=3-4 for ileum for T-bet; *P=0.005, n=7 for colon, *P=0.047, n=3-4 for ileum for Foxp3; *P=0.03, n=7 for colon, *P=0.006, n=3-4 for ileum for CD8+). Scale bar 100 μm. HPF: high power field. 40× objective. HPF=0.237 mm². Data in bar graphs are presented as mean±SEM.

FIG. 2 shows in one embodiment Intestinal immune cells influencing glucose homeostasis. (A) Absolute cell counts, including CD45+ (top far left), CD3+ (top middle left), CD3+CD4+ or CD3+CD8+ (top far right), CD4+ subsets (bottom far left), IFNγ+ CD8+ (bottom middle), and γδ+ T cell subsets (bottom far right) from colon and small bowel (SB) lamina propria after 12 weeks of HFD feeding in WT and Beta7^{null} (Beta7^{null}) mice. Entire colons were processed, or the distal 10 cm of SB (jejunum+ileum). (*P=0.0008 for CD45 colon, *P<0.0001 for CD45 SB; *P=0.045 for CD3 colon, *P=0.0004 for CD3 SB; *P=0.009 for CD4 colon, *P=0.0006 for CD4 SB, *P<0.0001 for CD8 SB; *P=0.02 for γδ SB; *P=0.0008 for CD4 IFNγ colon; *P=0.008 for CD4 IFNγ SB; *P=0.03 CD8 IFNγ SB; *P=0.001 for γδ IL-17 colon, n=4 experiments, 8-11 mice). (B) Body weights of WT and Beta7^{null} mice fed HFD over time, starting at 6 weeks of age (n=13 WT, n=11 Beta7^{null} mice). (C) Fasting glucose (left), GTT (middle) and ITT (right) of 12 week HFD-fed WT and Beta7^{null} mice (*P<0.05, n=13-15 WT, n=7-9 Beta7^{null} mice). (D) Food intake (left), and metabolic cage analysis, including oxygen consumption (left middle), carbon dioxide production (right, middle) and respiratory exchange ratio (RER) (right) of HFD WT and Beta7^{null} mice (n=7 for food intake, n=7 WT and n=6 Beta7^{null} mice for metabolic cage analysis). (E) Relative fat cell diameter (left) of mice, or number of VAT “crown-like structures” (CLS) per 100× low power field (right), after 12 weeks of HFD (*P<0.0001, fields counted from n=3 mice). Data in bar graphs are presented as mean±SEM.

FIG. 3 demonstrates ASA improving systemic metabolic parameters during HFD feeding. (A, left) Body weights of HFD and HFD 5-ASA-(1500 mg/kg/day) fed C57BL/6 mice over time, starting at 6 weeks of age (n=10). (A, right) VAT weights of mice, after 14 weeks of HFD or HFD 5-ASA feeding (1500 mg/kg/day) (n=10). (B, left) Relative fat cell diameter of mice, or (B, right) number of VAT “crown-like structures” (CLS) per 100× low power field, after 14 weeks of HFD or HFD 5-ASA feeding (1500 mg/kg/day) (n=3). (C) Fasting glucose (left), fasting insulin (right), (D) glucose tolerance test (GTT, left), insulin tolerance test (ITT, right) of mice after 14 weeks of HFD or HFD 5-ASA feeding (1500 mg/kg/day) (*P=0.001 for glucose, n=10 mice, *P=0.02 for insulin, n=8-9 mice, *P<0.05 for tolerance testing, n=10 mice for GTT, n=13-15 mice for ITT). (E) Fold change of pAkt/Akt protein ratios in mice fed HFD 5-ASA mice relative to HFD-fed controls (*P≤0.01, n=4 mice for VAT, 3-4 mice for liver and muscle). (F) Body weights, (G, left) GTT, (G, right) ITT after 8 weeks of HFD or HFD 5-ASA (1500 mg/kg/day) in mice switched over from 8 weeks of HFD (*P<0.05, n=5 mice, GTT was performed with an i.p. glucose challenge at a dose of 1.0 g/kg). Data in bar graphs are presented as mean±SEM.

FIG. 4 demonstrates in one embodiment ASA improving gut and VAT inflammation in mice during HFD feeding. (A) Intracellular staining of cytokines and Foxp3 in lamina propria T cell populations in the colons or (B) small bowel of mice after 16 weeks of HFD or HFD 5-ASA feeding (1500 mg/kg/day) (*P=0.001 for Th1, *P=0.01 for CD8, *P=0.045 for γδ T cell IL-17, n=2-3 experiments, 9 mice, for colon; *P=0.01 for Th1, *P=0.02 for CD8 IFNγ, *P=0.005 for Treg, *P=0.03 for γδ T cell IFNγ, *P<0.0001 for γδ T cell IL-17, n=2-4 experiments, 6-8 mice, for small bowel). (C) Flow cytometric analysis of T cell and (D) M1 macrophage subset in VAT of mice after 16 weeks of HFD or HFD 5-ASA feeding (1500 mg/kg/day) (*P=0.03 for Th1, *P=0.02 for CD8, *P=0.03 for Treg, *P=0.01 for macrophages, n=2 experiments, 8 mice). Data in bar graphs are presented as mean±SEM.

FIG. 5 shows in one embodiment 5-ASA targeting adaptive gut immunity in a PPARγ-dependent manner during HFD feeding. (A) Body weights, (B) GTT (left), ITT (right) of Rag1^{null} mice after 8 weeks of HFD or HFD 5-ASA feeding (1500 mg/kg/day) (n=4-5 mice). (C) Body weights of Beta7^{null} mice after 14 weeks of HFD or HFD 5-ASA feeding (n=15 HFD Beta7^{null}, n=11 HFD 5-ASA Beta7^{null}). (D) Fasting glucose (left), GTT (middle) and ITT (right) of Beta7^{null} mice after 12 weeks of HFD or HFD 5-ASA (n=9 HFD Beta7^{null}, n=10 HFD 5-ASA Beta7^{null}). (E) PPARγ mRNA expression of small bowel (SB) T cells compared to splenic T cells isolated from HFD-fed C57BL/6 mice (*P=0.004, n=3 mice). (F) PPARγ transcription factor activity of SB T cells from HFD or HFD 5-ASA-fed mice (*P=0.04, n=4 mice, normalized to total nuclear protein). (G, left) Levels of secreted IFNγ cytokine from small bowel (SB) T cells (left) or splenic (SP) T cells (right) from HFD-fed WT mice compared to HFD-fed Lck-Cre+ PPARγfl/fl mice treated with the indicated doses of 5-ASA in vitro (*P≤0.02 at all doses of 5-ASA for small bowel T cells, n=3 mice). (H) Levels of secreted IFNγ cytokine from OT-II T cells stimulated with 5-ASA-treated (0.1 or 1.0 mM) or untreated splenic (left) or small bowel (right) dendritic cells presenting the indicated concentrations of OVA323-339 peptide (*P≤0.03, n=3 samples, 3 spleens; n=2 samples, 4 pooled bowels). Data in bar graphs are presented as mean±SEM.

FIG. 6 shows in one embodiment 5-ASA and reduced gut inflammation improving intestinal barrier function and oral tolerance during HFD feeding. (A) Plasma FD4 concentration of age-matched NCD WT, HFD WT, HFD 5-ASA WT mice, and HFD Beta7^{null} mice after 12-16 weeks of diet following gavage as a measure of intestinal permeability (*P=0.02 for NCD Control vs HFD Control, *P=0.04 for HFD Control vs HFD 5-ASA, and *P=0.04 for HFD Control vs. HFD Beta7^{null}; n=10 NCD Control, n=10 for HFD Control, n=8 HFD 5-ASA, and n=6 HFD Beta7^{null} mice). (B, far left) Serum anti-LPS IgG levels of age-matched NCD WT, HFD WT, HFD 5-ASA WT, and HFD Beta7^{null} mice after 14 weeks HFD feeding (*P≤0.03, n=5-8). (B, middle and far right) Serum endotoxin levels (middle) and VAT endotoxin levels (right) of age-matched NCD WT, HFD WT, and HFD 5-ASA WT after 14 weeks HFD feeding (*P<0.05 for serum endotoxin, P=0.19 for VAT endotoxin; n=3-4 for serum endotoxin, n=5 for VAT endotoxin). (C, left) Plasma FD4 concentrations, following oral gavage, of age-matched IFNγ^{null} mice after 10 weeks of HFD feeding (*P=0.01, n=4 mice). (C, right) ZO-1 mRNA expression relative to housekeeping gene expression in MODE-K intestinal cells treated with indicated amounts of IFNγ in vitro (*P=0.006, n=3 in each treatment). (D) Ratio of OVA-specific IgG1/IgG2c (left) and OVA-specific IgA (right) in age-matched NCD WT, HFD WT, and HFD 5-ASA WT mice 2 weeks after immunization with OVA-CFA (*P=0.03 for IgG1/IgG2c, *P=0.03 for oral NCD vs oral HFD IgA, *P=0.02 for oral HFD vs oral HFD 5-ASA IgA; n=4-6 mice). (E) OVA-specific recall IL-2 and IFNγ responses in age-matched HFD WT, and HFD 5-ASA WT mice from axillary lymph nodes, 2 weeks after immunization with OVA-CFA (*P=0.01 for IL-2, *P=0.006 for IFNγ; n=4-5 mice in duplicates). (F) OVA tetramer-stained Treg cells from VAT of age-matched oral or non-oral challenged HFD WT, and HFD 5-ASA WT mice 2 weeks after immunization with OVA-CFA. (*P=0.03; n=2 experiments, 6 pooled mice). Data in bar graphs are presented as mean±SEM.

FIG. 7 (A-B) shows in one embodiment impact of short-term (3 weeks) HFD feeding on T cell populations in colon and small bowel. Percentages of IFNγ- and IL-17-producing CD4+ T cells (left), IFNγ-producing CD8+ T cells (second from left), CD4+Foxp3+ regulatory T cells (Tregs, third from left) and IFNγ- and IL-17-producing γδ T cells (far right) of (A) colons and (B) small bowels of age-matched mice fed NCD vs HFD for 3 weeks (n=2-3 for colon, 5 pooled mice, and n=4-5 for small bowel, *P=0.04 for Treg in A, *P=0.02 for IL-17-producing γδ T cells). (C-D) Absolute counts of intestinal immune cells after 12-16 weeks of HFD. (C) Absolute counts of cytokine-producing CD4+ and CD8+ T cells, CD4+Foxp3+ Tregs, and γδ T cells from colon lamina propria after 12-16 weeks of HFD feeding (*P<0.05 for IFNγ-producing CD4+ T cells, *P=0.003 for IL-17-producing γδ T cells, n=2-3 experiments, 4-6 mice). (D) Absolute counts of cytokine-producing CD4+ and CD8+ T cells, CD4+Foxp3+ Tregs, and γδ T cells from small bowel lamina propria after 12-16 weeks of HFD (*P=0.04 for IFNγ-producing CD4+ T cells, *P=0.02 for IFNγ-producing CD8+ T cells, *P=0.03 for Treg. n=5-8, except for Tregs where n=3). HFD alters ILC numbers in the absence of histological changes. (E) Absolute numbers of innate lymphoid cells (ILCs) per colon in NCD and HFD mice (n=3, *P<0.05) (left) and proportion of ILC subsets (right), gated as described (Kirchberger, S. et al. 2013) (n=3, *P<0.05).

FIG. 8 (A-B) shows in one embodiment percentages of intestinal and splenic immune cells in HFD WT and Beta7^{null} mice, including CD3+ subsets (left), CD4+ T cell subsets (second from left), IFNγ-producing CD8+ T cells (third from left) and γδ T cell subsets (right) from (A) colon and small bowel (SB) lamina propria and (B) spleens after 12 weeks of HFD. Entire colons were processed, or the distal 10 cm of SB (jejunum+ileum), (*P=0.016; n=3-5). Absolute cell counts of (E) CD45+ cells, (F) T cells, including (G) CD3+CD4+ or CD3+CD8+ cells, (H) CD4+ T cell subsets and (I) CD8+ T cells from VAT after 12 weeks of HFD feeding in WT and Beta7^{null} mice. (J) Absolute counts (left) and percentages (right) of M1 macrophages in the VAT. (*P=0.003 for CD45+, *P=0.009 for CD3+, *P=0.004 and 0.017 for CD4+ and CD8+, respectively; *P=0.007 for CD11c+CD206− macrophage counts and *P=0.02 for percentages; n=2 and 3, respectively). (K) Reconstitution of HFD Rag1^{null} mice, spleen (left) and VAT (right) by WT vs Beta7^{null} splenic T cells (n=6). (L-M) Metabolic parameters of NCD WT and NCD Beta7^{null} mice. (L) Body weights of NCD WT and NCD Beta7^{null} mice at 18 weeks of age. (M) Fasting glucose (left), glucose tolerance test (GTT, middle) and insulin tolerance test (ITT, right) of NCD WT and NCD Beta7^{null} mice at 18 weeks of age (*P<0.05, n=7 WT, n=7 Beta7^{null} mice). Data in bar graphs are presented as mean±SEM.

FIG. 9 shows in one embodiment (B) Organ weights and (C) gluconeogenesis gene expression of HFD or HFD 5-ASA mice. (D) Q-PCR analysis of VAT (left) or SAT (right) lysates for levels of adipogenesis genes expressed in mice fed HFD 5-ASA relative to HFD control after 18 weeks of diet (n=10 control, n=10 treated). (E) Food intake, (F) oxygen consumption (left), carbon dioxide output (middle) and RER (right) after 14 weeks of either HFD or HFD 5-ASA (n=5). (G) Body weights, (H) GTT (left) and ITT (right) after 24 weeks of HFD or HFD mixed with low dose 5-ASA (150 mg/kg/day), (n=5, *P<0.05). (I) Body weights (far left), fasting glucose (middle left), GTT (1.5 g/kg, middle right), and ITT (far right) of C57BL/6 mice after 12 weeks of either conventional NCD or NCD 5-ASA (1500 mg/kg/day) diet (n=5-10 NCD mice, 4-9 NCD 5-ASA mice). Data in bar graphs are presented as mean±SEM.

FIG. 10 (A) shows in one embodiment proportions of Th1 T cells and Th17 T cells (left), IFNγ-producing CD8+ T cells (middle), and CD4+Foxp3+ Tregs (right) in the spleens of mice fed 14 weeks HFD or HFD 5-ASA (n=2). (B) Cytokine production, either IL-10 or IFNγ, of cultured splenocytes after 48 hrs with plate-bound anti-CD3/CD28 (n=3). (C) Proportions of Th1 T cells, and Th17 T cells (left), IFNγ-producing CD8+ T cells (middle) and CD4+Foxp3+ Tregs (right) in the peripheral blood of mice fed 14 weeks HFD or HFD 5-ASA (n=5). (D) Luminex analysis of 23 cytokines in serum of mice after 14 weeks of HFD or HFD 5-ASA (n=10, *P<0.05). (E) Concentrations of 5-ASA in serum (left), colon, small bowel (SB), or VAT (right) as measured by HPLC against internal 4-ASA control, after 14 weeks of HFD or HFD 5-ASA (n=2-3) (′ND′ indicates non-detectable concentrations). Data in bar graphs are presented as mean±SEM.

FIG. 11 (A-D) shows in one embodiment impact of 5-ASA treatment on intestinal immune populations in NCD-fed mice. Percentages IFNγ and IL-17-producing CD4+ T cells (left), IFNγ-producing CD8+ T cells (second from left), CD4+Foxp3+ Tregs (third from left), and IFNγ- and IL-17-producing γδ T cells (far right) of (A) colons (n=4), (B) small bowels (n=2-4), (C) VAT (n=1-4), and (D) spleens (n=2-4) of NCD- vs NCD 5-ASA-fed mice. Data in bar graphs are presented as mean±SEM. PPARγ agonism decreases IFNγ secretion in activated small bowel T cells. (E) PPARγ mRNA expression of small bowel (SB) T cells compared to splenic T cells isolated from NCD-fed C57BL/6 mice (*P=0.0024, n=5-9 mice). (F) Levels of secreted IFNγ cytokine from small bowel T cells (left) or splenic T cells (right) from HFD-fed WT mice co-cultured with 5-ASA (0.1 mM), rosiglitazone (ROSI, 0.1, 1 and 10 μM), or combination of the two (ROSI+5-ASA). (*P=0.036 for 5-ASA, *P=0.013 for ROSI 0.1, *P=0.009 for ROSI 1, *P=0.002 for ROSI 10, *P=0.02 for ROSI 0.1+5-ASA, *P=0.0006 for ROSI 1+5-ASA, and *P=0.0008 for ROSI 10+5-ASA as compared to untreated control, n=3-4 mice).

### DETAILED DESCRIPTION

Generally, the present disclosure provides compounds and uses to treat patients with high blood glucose levels in order to help regulate glucose levels. In some embodiments high blood glucose is determined using a fasting plasma glucose test. In some embodiments high blood glucose is determined using an oral glucose tolerance test (OGTT). In some embodiments high blood glucose is determined using a random plasma glucose estimate. In some embodiments, high blood glucose is determined using the A1C test (e.g. glycosylated hemoglobin test). In some embodiments, high blood glucose is determined as a blood glucose level above normal. In some embodiments the fasting plasma glucose level is between about 6.1 and 6.9 mmol/L (which may be characterized as pre-diabetes). In some embodiments the fasting plasma glucose level is ≥7 mmol/l. In some embodiments, the oral glucose tolerance test is utilized to measure blood glucose, and the 2-hour plasma glucose (2hPG) level in a 75 g OGTT is between about 7.8 and 11.9 mmol/L. In some embodiments, the 2-hour plasma glucose (2hPG) level in a 75 g OGTT is of ≥11 mmol/L. In some embodiments the A1C level is between about 6.0 and 6.4 percent. In some embodiments the A1C level of ≥6.5%. In some embodiments that random plasma glucose level is ≥11 mmol/L.

In some embodiments high blood glucose levels are as a result of glucose intolerance. In some cases high blood glucose levels are as a result of insulin resistance. In some embodiments high blood glucose levels are as a result of Type 1 diabetes, Type 2 diabetes and/or obesity. In other embodiments high blood glucose levels are as a result of glucose intolerance which is itself a result of Type 1 diabetes, Type 2 diabetes and/or obesity. In other embodiments high blood glucose levels are as a result of insulin resistance which is itself a result of Type 2 diabetes and/or obesity.

In some embodiments, patients with high blood glucose levels can receive benefit from being treated with a gut specific anti-inflammatory agent. In some embodiments, the gut specific anti-inflammatory agent is a locally gut active anti-inflammatory agent. In some embodiments, the gut specific anti-inflammatory agent is 5-ASA, or derivative, analogue or prodrug is selected from the list of mesalamine, sulfasalazine, asacol, delzicol, pentasa, lialda, apriso, and olsalazine. In some embodiments the gut specific anti-inflammatory agent or locally gut active anti-inflammatory agent is a PPAR gamma modulator. In some embodiments the PPAR gamma modulator is balsalazide or GED-0508-34.

One well known locally active, gut-specific anti-inflammatory agent is mesalamine (5-ASA), the first line maintenance therapy for inflammatory bowel disease (IBD) for over 30 years (Rousseaux et al., 2005). 5-ASA is a salicylic acid derivative with anti-inflammatory properties that acts locally in the gut with minimal systemic absorption and side effects. As IBD is also characterized by increased intestinal inflammation and altered permeability (Brown et al., 2013), we hypothesized that other gut-specific, or locally gut active anti-inflammatory agents might have beneficial effects in treating high blood glucose, and may help elucidate the role of gut immune cells in this disease.

### Methods

Mice. Mice were fed either NCD (15 kcal % fat) or HFD (Research Diets, 60 kcal % fat, irradiated) starting at 6 weeks of age. All studies were performed under the approval of Animal User Protocols by the Animal Care Committee at the University Health Network. Mice were maintained in a pathogen-free, temperature-controlled environment on a 12-hour light and dark cycle. All mice used in comparative studies were male, age-matched, and litter mates where possible. For 5-ASA diet studies, age-matched mice were randomly assigned to 5-ASA diet or to control diet in groups of 5 mice per cage. We confirmed T cell-specific floxing of the PPARγ gene in Lck-Cre+ PPARγfl/fl mice by qPCR with a minimum of at least 90% reduction in PPARγ expression.

Compounds and Treatment Diets.

5-aminosalicyclic acid powder (Sigma-Aldrich) was incorporated directly into the HFD at two doses (150 mg/kg/day and 1500 mg/kg/day), corresponding to the equivalent human dosage of 720-7200 mg/day, by Research Diets Inc. 5-ASA was mixed into NCD at 1500 mg/kg/day by Harlan Laboratories.

Metabolic Cage Studies.

We placed mice in automated metabolic cages (Oxymax Systems, Columbus Instruments) for 48 hours with airflow held constant at 0.5 L/min. (Revelo et al., 2014). We placed mice in automated metabolic cages (Oxymax Systems, Columbus Instruments) for 48 h with airflow held constant at 0.5 L/min. We measured metabolic activity using indirect calorimetry, recording maximal O₂ consumption (VO₂), CO₂ production (VCO₂), and heat production normalized to body weight. Respiratory exchange ratio (RER) was calculated as VCO₂NO₂. The data shown are calculated for light and dark measurements as an average over 24 hours by combining light and dark measurements. Ambulatory activity was measured by the breaking of infrared laser beams in the XY plane.

Metabolic Studies.

We measured body weights, GTTs, ITTs, serum insulin, and fat cell diameter as previously described (Winer et al., 2009a). All GTTs were performed with a 1.5 g/kg glucose i.p. injection unless indicated otherwise.

Isolation of bowel immune cells. For isolation of small intestine lamina propria immune cells, we used the protocol described by Fritz et al (Fritz et al., 2012), and processed approximately 10 cm from the distal end of the small intestine (jejunum and ileum). For the isolation of colonic lamina propria immune cells, we used the protocol described by Geddes et al (Geddes et al., 2011). Following lamina propria isolations, we passed immune cells through 70 μm strainers, and used them for flow cytometry.

Histology.

We fixed VAT, colons and small bowel ileums, from mice for 48 h in 10% buffered formalin before processing and hematoxylin/eosin staining. We enumerated crown-like structures (CLSs) in VAT by counting the number of adipocytes completely surrounded by immune cells identified on hematoxylin/eosin staining per 100× low power field. Analysis of histochemical stains was performed in a blinded fashion by two certified pathologists (S.W. and D.W.).

Isolation of VAT and Bowel-Associated Immune Cells.

We isolated VAT-associated immune cells as previously described (Winer et al., 2009a).

Flow Cytometry.

We stained single cell suspensions for 30 min on ice with commercial antibodies. Flow cytometry antibodies including CD45.2 (104), CD3 (145-2C11), CD4 (GK1.5), CD8 (53-6.7), CD25 (PC61), γδTcR (GL3), Foxp3 (150D), IL-17 (TC11-18H10.1), IFNγ XMG1.2), α4β7 (DATK32), CCR9 (CW-1.2), CD11b (M1/70), F4/80 (BM8), CD11c (N418), CD206 (C068C2), Gr-1 (RB6-8C5), IL-7Rα (A7R34), B220 (RA3-6B2), Thy1.2 (30-H12), NKp46 (29A1.4), and Sca-1 (D7) were purchased from Biolegend. Intracellular staining was performed using a Foxp3 staining buffer kit (eBioscience). We acquired data on a Fortessa flow cytometer (BD Biosciences) and analyzed it with FlowJo software (Tree Star). We gated ILCs as described (Kirchberger et al., 2013).

Human Bowel Samples and Immunohistochemistry.

We obtained colon and small bowel samples from histologically normal margins of surgical resection specimens for patients with sporadic colon cancer at the Toronto General Hospital. Patients characterized as lean demonstrated BMIs of 21.3±0.5, while obese BMIs were 34.2±1.7. For immunohistochemistry, we used antibodies against FoxP3 (Abcam), T-bet (Epitomics), CD8 (clone 4b11) (Vector Labs). Double staining: paraffin embedded sections were subjected to pH 9 Tris-EDTA antigen retrieval in a heated pressure cooker. Primary antibodies were used at the following dilutions: 1 in 400 rabbit monoclonal antibody to T-bet (Epitomics), and 1 in 100 mouse monoclonal antibody to Foxp3 (Abcam). Primary antibodies were detected with a Mach2 double stain-2 kit (Biocare Medical) and color was produced with Vector Red and diaminobenzidine (Vector labs). Immunohistochemistry analysis was performed in a blinded fashion. We calculated T-bet and Foxp3 ratios by taking the average of at least 10 HPF per patient. See Table S1.

TABLE S1
Summary of relevant clinical characteristics
of patients used in histology studies

| | Obese (n = 7) | Lean (n = 7) | P-value |
|---|---|---|---|
| Average Random | 6.87 ± 0.22 | 5.77 ± 0.17 | 0.0018 |
| Glucose (mmol/L) | | | |
| Age | 64.4 ± 2.1 | 66.5 ± 2.8 | 0.55 |
| Gender | (3M:4F) | (5M:2F) | 0.59^{a} |
| BMI (kg/m²) | 34.2 ± 1.7 | 21.3 ± 0.5 | <0.0001 |
| Distance to tumour - colon (cm) | 13.3 ± 2.6 | 9.6 ± 1.7 | 0.25 |
| Distance to tumour - small | 23.9 ± 9.5^{b} | 9.1 ± 5.8^{b} | 0.22 |
| intestine (cm) | | | |
| Confounding medications | | | |
| (# of patients): | | | |
| Neoadjuvant capecitabine | 1/7 | 0/7 | 1.00^{a} |
| Statins | 6/7 | 3/7 | 0.26^{a} |
| Aspirin - 81 mg dose | 4/7 | 2/7 | 0.59^{a} |
| Antidiabetic drugs | 0/7 | 0/7 | 1.00^{a} |
| PPARγ agonist | | | |
| Insulin | | | |
| Metformin | | | |
| Glyburide | | | |
| GLP-1 agonist | | | |

^{a}analyzed by Fisher's exact test
^{b}n = 3 and 4 obese and lean patients provided small intestine samples, respectively.

Western Blotting.

We injected mice i.p. with insulin (1.5 U/kg) or PBS and harvested tissues after 10 min. We probed tissue lysates for phospho-Akt (S473), total Akt and GAPDH (Cell Signaling Technology). We snap-froze tissues in liquid nitrogen. To make tissue lysates, we mechanically homogenized VAT, liver and muscle tissues in ice-cold lysis buffer (Santa Cruz) and centrifuged them at 14,000×g for 10 min at 4° C. Supernatants were collected and separated by SDS-PAGE and subjected to blotting with indicated antibodies.

PPARγ Activity Assay.

We measured PPARγ functional activity in T cell nuclear extracts using a PPARγ transcription factor binding assay following vendor's instructions (ThermoScientific and Cayman Chemical Company). This ELISA-based assay is precoated with dsDNA containing the peroxisome proliferator response element (PPRE). PPARγ in the isolated nuclear extracts bind to PPRE. As per vendor's recommendations, this assay is specific to PPARγ and not to other PPARs (i.e., α or δ).

In Vitro Co-Culture Studies.

We purified splenic or small bowel T cells, or dendritic cells and treated with 5-ASA at indicated concentrations. To measure the effect of IFNγ on tight-junction gene expression, we treated MODE-K cells with recombinant mouse IFNγ (Biolegend). We purified bulk dendritic cells and T cells from murine small bowels and spleens using a negative selection DC or T cell isolation kit (Stem Cell Technologies). We plated T cells at 5×10⁵ cells/well with plate-bound anti-CD3/CD28 (1 μg/ml, Biolegend). We dissolved 5-ASA in culturing media (pH 7.3) and sterile-filtered the solution. Dissolved 5-ASA was added to designated wells at concentrations of 0, 0.01, 0.1, and 1 mM. We collected supernatants for cytokine measurements after 72 hour incubation. In rosiglitazone experiments, we added varying concentrations of rosiglitazone (0.1, 1 and 10 μM) in culturing media either alone or with 0.1 mM 5-ASA. For DC-T cell co-culture experiments, we pre-treated DCs with 0, 0.1, or 1.0 mM 5-ASA. After 24 hours, we washed the cells with PBS and co-cultured them (1×10⁴ cells/well) with OVA323-339 peptide and OT-II splenic or small bowel T cells (5×10⁴ cells/well) for 48 hours. Supernatants were collected at 48 hours for cytokine measurement.

MODE-K Cell Line.

The murine intestinal epithelial cell line derived from C3H/He mice. These cells were propagated under standard protocol using DMEM (Gibco) containing 10% FBS, 10 mM HEPES, 50 μM 2-Mercaptoethanol, 50 mg/mL Streptomycin and 50 U/ml Penicillin. In MODE-K in vitro studies, we split cells at 70-80% confluency and seeded at a density of 3×10⁵/well for treatment with recombinant mouse IFNγ (Biolegend) (5 or 10 ng/mL) for 24 hours. We mechanically detached the cells for RNA isolation (Qiagen).

Gut Permeability Assays (FD4).

We measured gut permeability in overnight fasted mice 4 hours after oral gavage with 0.4 mg/g of FITC-conjugated dextran (Sigma) as described (Dong et al., 2014).

Endotoxin Measurements.

We measured endotoxin levels in the serum and adipose tissue using Pyrogene Recombinant C endotoxin detection fluorescence kit (Lonza Inc.). We measured mouse serum anti-LPS IgG antibody levels with a commercially available kit (Chrondrex Inc.).

Cytokine Measurements.

We measured serum cytokines by Luminex Multiplex cytokine assay (Millipore, run by the UHN Microarray Center), and quantified IL-10 and IFN-γ in supernatants of anti-CD3/CD28-stimulated splenocytes or bowel immune cells (Winer et al., 2009b) by ELISA (Biolegend).

Quantitative PCR (q-PCR).

We extracted total RNA from isolated or cultured cells using a RNeasy Mini Kit (Qiagen), and for subcutaneous adipose tissue (SAT) and VAT we used a RNeasy Lipid Extraction kit (Qiagen). We reverse-transcribed the RNA by random primers with M-MLV (Invitrogen). We performed q-PCR with a 7900HT PCR system (Applied Biosystems) using SYBR® Green master mix reagent (Applied Biosystems). We assessed expression of adipocyte P2 (aP2), CCAAT/enhancer-binding protein-α (CEBPα), Peroxisome proliferator-activated receptor-γ (PPARγ), and sterol regulatory element-binding protein (SREBP) cDNA. Each sample (n=5 Control, n=4 HFD 5-ASA) was run in triplicate and normalized to housekeeping genes, 18s or GAPDH. We calculated relative fold changes in gene expression normalized to 18s or GAPDH by the ΔΔCT method using the equation 2-ΔΔCT. The results are shown as fold changes compared to the control group. See Table S2.

TABLE S2
Primer sequences used for quantitative RT-PCR

| Primer | Sequence |
|---|---|
| 18s forward | AGTCCCTGCCCTTTGTACACA |
| 18s reverse | CGATCCGAGGGCCTCACTA |
| GAPDH forward | TCACCACCATGGAGAAGGC |
| GAPDH reverse | GCTAAGCAGTTGGTGGTGCA |
| FABP4 forward | GACGACAGGAAGGTGAAGAG |
| FABP4 reverse | ACATTCCACCACCAGCTTGT |
| CEBPα forward | AAGAACAGCAACGAGTACCGG |
| CEBPα reverse | CATTGTCACTGGTCAGCTCCA |
| SREBP-1C forward | GATCAAAGAGGAGCCAGTGC |
| SREBP-1C reverse | TAGATGGTGGCTGCTGAGTG |
| PPARγ forward | GCCCTTTGGTGACTTTATGG |
| PPARγ reverse | CAGCAGGTTGTCTTGGATGT |
| G6pc forward | TCTGTCCCGGATCTACCTTG |
| G6pc reverse | GTAGAATCCAAGCGCGAAAC |
| Pck1 forward | GTGAGGAAGTTCGTGGAAGG |
| Pck1 reverse | TCTGCTCTTGGGTGATGATG |
| Zo-1 forward | GCCGCTAAGAGCACAGCAA |
| Zo-1 reverse | TCCCCACTCTGAAAATGAGGA |

Oral Tolerance Studies.

We administered oral ovalbumin (OVA) in the form of drinking water (1 mg/mL) for 7 days to NCD-, HFD- or HFD 5-ASA-fed mice to induce oral tolerance. Average consumption was similar across different groups, at 5 mL per day. Following oral tolerance induction, we immunized animals with or without oral tolerance induction subcutaneously with OVA/CFA (50 μg per side) on both sides of the chest. Axillary draining lymph nodes and VAT were collected 7 days following immunization and subjected to immunological analysis, including OVA323-339/I-A^{b} tetramer staining and OVA-induced cytokine production and proliferation in vitro. Briefly, we processed axillary lymph nodes into single-cell suspension through a 70 μm filter and cultured the cells (5×10⁵/well) in 96-well U-bottom plate with indicated concentrations of OVA (Sigma). We collected culture supernatants at 48 h for determination of IL-2 and IFNγ via ELISA (Biolegend). To measure OVA-specific antibody responses, we performed ELISA by plating diluted serum samples (1:200-1:4000) onto OVA-coated ELISA plates (10 μg/mL), followed by biotinylated anti-mouse isotype-specific secondary antibodies (1:5000 dilution) (Southern Biotech) and HRP-conjugated streptavidin (Biolegend). To measure-OVA-specific Tregs, we stained VAT immune cells with a 1:200 dilution of PE-conjugated OVA323-339/I-A^{b} or control hCLIP/I-A^{b} tetramers (NIH Tetramer Core Facility) in FBS-supplemented RPM-I for 4 hours at 37° C., followed by antibody staining for other surface antigens and Foxp3.

Bowel Trafficking Experiments.

We injected WT or Beta7^{null} splenic T cells (5×106) i.p. into Rag1^{null} mice fed a HFD for 16 weeks. After 48 hours, we analyzed spleens and VAT stromal vascular fraction for percentages of CD3+ T cells by flow cytometry.

HPLC Sample Preparation.

All chemicals including 4-aminosalicylic acid (4-ASA) were purchased from Sigma Chemicals. All reagents were of HPLC grade and purchased from Caledon Labs. We cut frozen tissues and homogenized them in 80% methanol (30 mg/mL) on ice. We spiked 100 μL aliquots with 4-ASA used as internal standard (200 ng), and 400 μL 80% methanol were added. For plasma, we thawed samples from −80° C. at room temperature. 75 μL plasma were spiked with 4-ASA used as internal standard (200 ng) and mixed with 300 μL of methanol for 5 minutes (vortex). Samples were centrifuged (20,000×g, 15 minutes, 4° C.). Supernatants were separated from the pellets and taken to dryness under nitrogen gas in the fumehood. We then reconstituted the dry residues in 200 μL of mobile phase, vortexed for 1 minute and centrifuged at 20,000×g for 15 minutes. The supernatants were transferred to autosampler vials fitted with inserts and sealed caps. Aliquots of the solution were injected into HPLC for analysis.

HPLC-Fluorescence-UV Analysis.

We performed high performance liquid chromatography using a Dionex Ultimate 3000 system equipped with a binary pump, a built-in autosampler, a photodiode array and a RF 2000 fluorescence detector. Chromatographic separation of the compounds was accomplished using a reverse phase Kinetec C18 column (5 μm, 150×4.6 mm) (Phenomenex Inc.) using a binary gradient mobile phase with 17.5 mM potassium phosphate buffer as solvent A (equal molar concentration of both monobasic and dibasic potassium salts at a pH of 3.50 adjusted by phosphoric acid) and methanol as solvent B as previously described (Hong et al., 2011). Samples were injected and the separation was performed at room temperature at a flow rate of 0.8 mL/min. The run time was 15 minutes. The analytes were monitored by fluorescence (excitation: 337 nm and emission: 432 nm) and by (UV235 nm). We analyzed the chromatograms produced using Chromeleon version 6.8 software.

Gut Microbiome Sequencing.

We amplified the V4 hypervariable region of the 16S rRNA gene using a universal forward sequencing primer and a uniquely barcoded reverse sequencing primer to allow for multiplexing (Caporaso et al., 2012). Primers contained an adapter sequence to bind the amplicons to the Illumina flow cell. PCR-based library construction was performed in triplicate 25 μl solutions containing 1×KAPA2G Robust HotStart ReadyMix, 600 nM each of primer, and 1 μl of DNA template. For every PCR reaction sterile dH₂O was used as a negative control to ensure no contaminating DNA was present. PCR conditions were 95° C. for 3 min, followed by 18 cycles of 95° C. for 15 s, 58° C. for 15 s, 72° C. for 15 s and were completed at 72° C. for 5 min. All PCR reactions were run on a 1% agarose gel to visualize the amplification and approximate DNA quantity. Individual barcoded samples from the triplicates were pooled by approximately even concentrations to create the final library. The final library was purified using 0.8 volumes of Agencourt AMPure XP beads (Beckman Coulter, Indianapolis, Ind.) according to the manufacturer's protocol and quantified using the Qubit Fluorometer. The final library was prepared according to the MiSeq user guide, diluted to a concentration of 7 pM and combined with a 5% PhiX control. Sequencing was performed using the V2 (150 bp×2) chemistry and sequenced on the Illumina MiSeq (Illumina, San Diego, Calif.).

Statistical Analyses.

Statistical significance between two means was assessed with an unpaired two sided t-test. In Figure legends describing experiments from pooled animal tissues, the number of biological experiments is listed as the n-value, followed by the total number of pooled mouse samples. All data are presented as means±SEM. Statistical significance was set at P<0.05.

The advantages of the present invention are further illustrated by the following examples. The examples and their particular details set forth herein are presented for illustration only and should not be construed as a limitation on the claims of the present invention.

### EXAMPLES

### Example 1

We demonstrate that diet-induced obesity is accompanied by a low-grade functional pro-inflammatory shift in lamina propria immune cell polarity, consistent with changes previously described in response to an intestinal barrier defect (Brown et al., 2013). Genetic reduction of inflammatory gut immune cells, using mice deficient in beta7 integrin, leads to improved glucose tolerance in diet-induced obese (DIO) mice. Treatment of DIO mice with 5-ASA reverses the pro-inflammatory shift in bowel immune cells, reduces VAT inflammation, and improves metabolic parameters. The mechanistic effects of 5-ASA are associated with reduced gut permeability, improved oral tolerance to soluble luminal-derived antigen, and increased luminal antigen-specific Tregs in VAT. These data demonstrate that the gut immune system is an important targetable component to the development of obesity-associated IR, and that gut-specific anti-inflammatories, including represent a new class of potentially effective, minimal side effect therapies for IR.'s

### Example 2

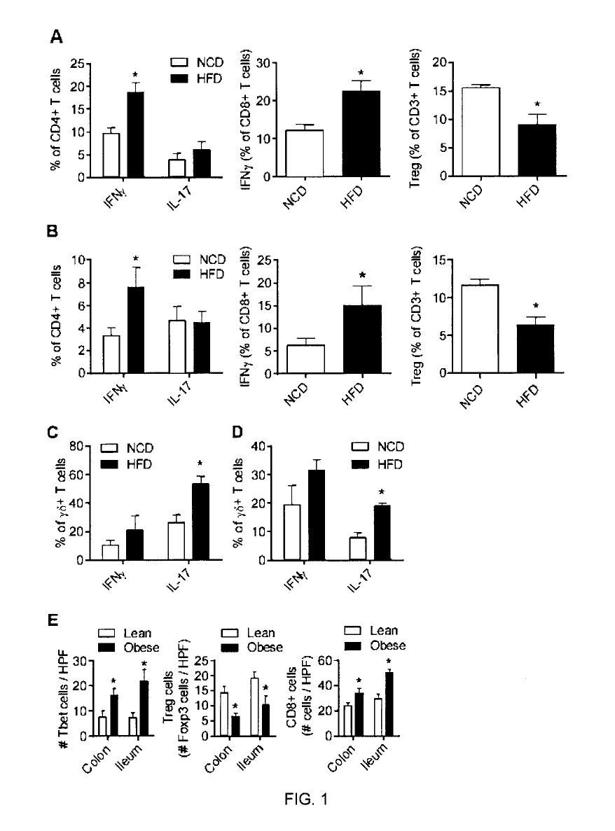

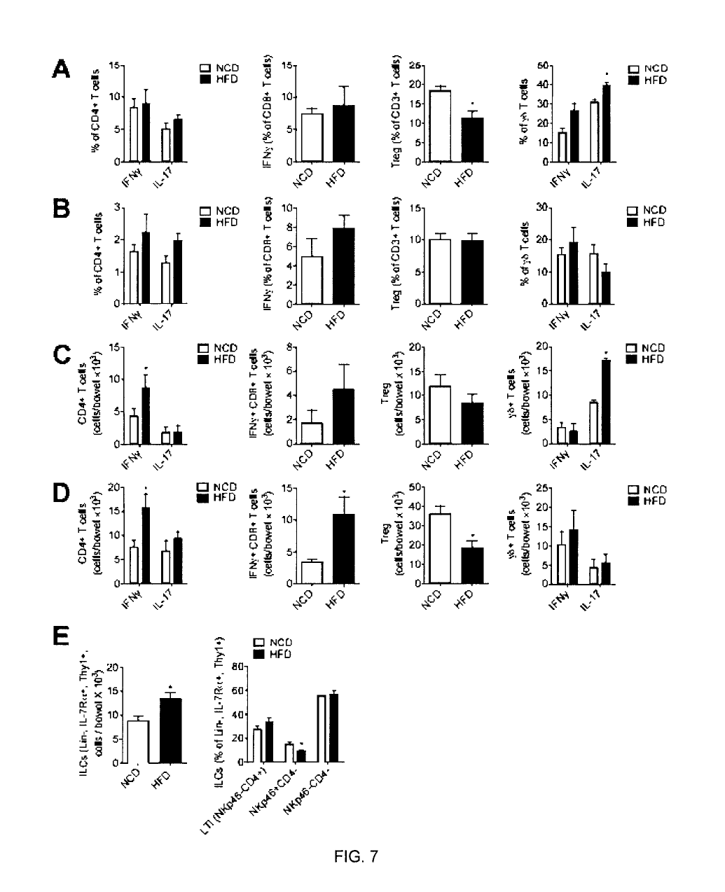

To determine the effects of diet-induced obesity on gut immunity, we investigated if adaptive immune cell populations in the colon and small bowel lamina propria are altered by HFD feeding in C57BL/6 mice at 3 or 12-16 weeks of HFD. After 3 weeks of HFD, changes in the proportions of bowel immune populations began in the colon and were characterized by a reduction in the percentage of Tregs and an increase in IL-17-producing γδ T cells (FIG. 7A-B). However, after 12-16 weeks of diet, HFD induced a pro-inflammatory shift in immune cells from both the colon and small bowel. In colonic immune cells, there was an increase in the proportion and/or absolute number of IFNγ-producing Th1 T cells and CD8+ T cells and a significant reduction in the proportion of CD4+Foxp3+ Tregs (FIG. 1A and FIG. 7C). In the small bowel of HFD mice, there was an increase in the frequencies and/or numbers of Th1 CD4+ T cells and IFNγ-producing CD8+ T cells and a significant decrease in the proportion and absolute number of CD4+Foxp3+ Tregs (FIG. 1B and FIG. 7D). We next assessed the effects of 12-16 weeks HFD feeding on γδ T cells and innate lymphoid cell populations in the bowel. HFD was associated with marked increases in the frequencies and/or numbers of IL-17-producing, but not IFNγ-producing γδ T cells in the colon and small bowel (FIGS. 1C and D, FIGS. 7C and D). Furthermore, there was an increase in total cell numbers of innate lymphoid cells in the colon, though the relative proportion of NKp46+CD4− cells was reduced in HFD-fed mice (FIG. 7E).

### Example 3

To determine if humans showed similar changes in gut immune populations with obesity, we correlated patient BMI with relative numbers of pro-inflammatory T-bet+ (Th1, ILC1 (Bernink et al., 2013)) T cells, anti-inflammatory Foxp3+ (Treg) T cells, as well as CD8+ T cells present in the lamina propria of colon and ileum resection specimens. Table S1 summarizes relevant clinical parameters of patients included in the study. Obese patients showed significant increases in colon and small bowel Tbet+ cells and CD8+ cells in addition to a reduction in Tregs (FIGS. 1E and F). We have demonstrated a reduction in gut Tregs and a pro-inflammatory shift in some adaptive and innate T cell populations in the gut of HFD-fed mice, with a similar observation in our specific cohort of obese humans. Interestingly, this inflammatory shift was not associated with any apparent histological changes of chronic or active inflammation on H&E stained sections of obese human or HFD-fed mouse colons.

### Example 4

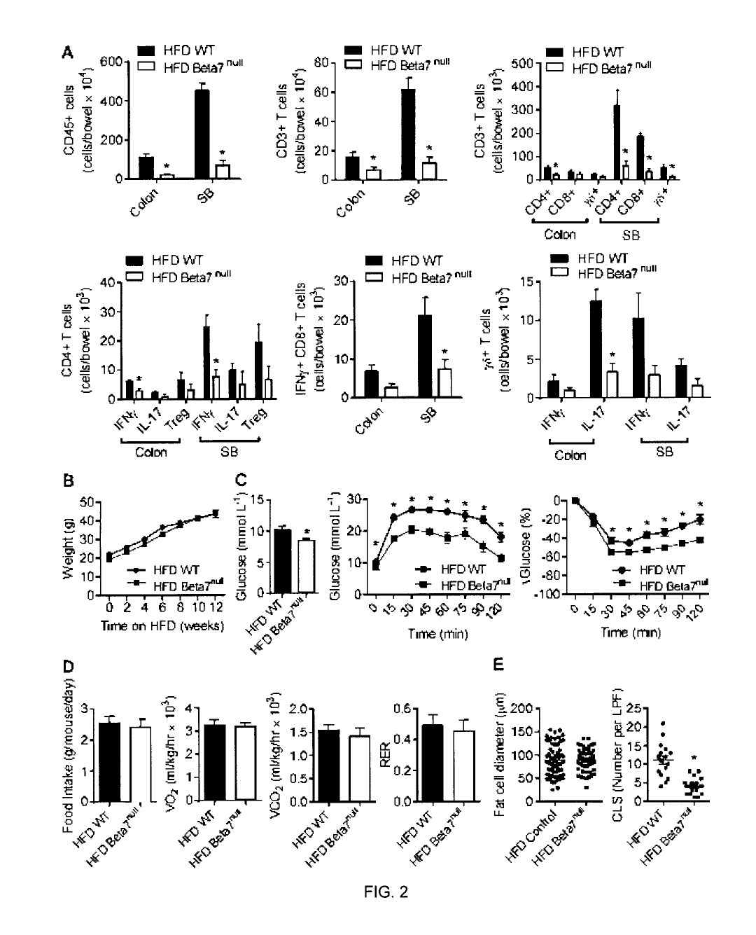

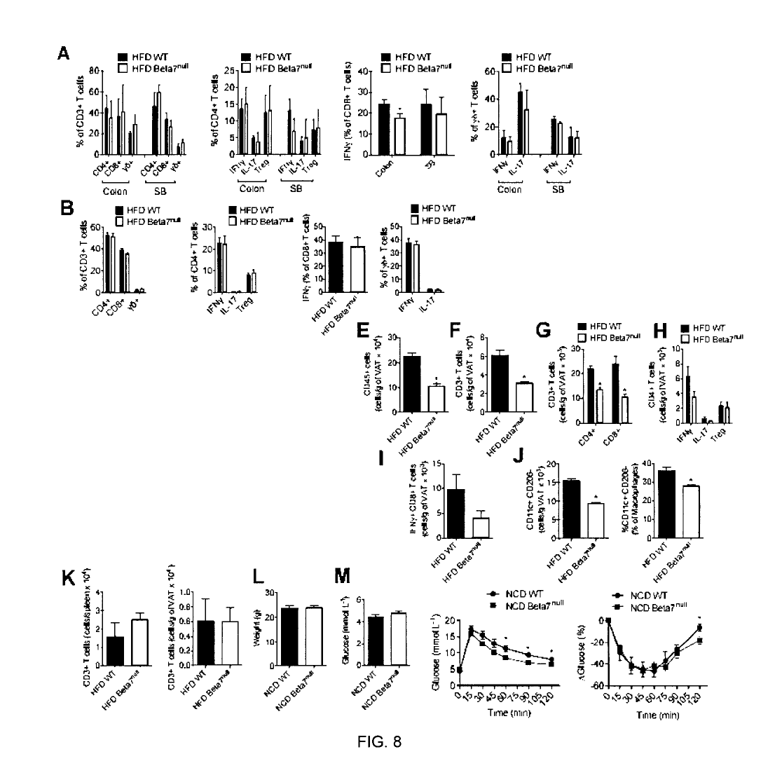

We next determined if the gut immune system as a whole could contribute to the development of obesity-associated IR. To address this issue, we placed beta7 integrin-deficient C57BL/6 mice (Beta7^{null} mice) on either normal chow diet (NCD) or HFD for 12 weeks and then assessed metabolic parameters. Beta7 pairs with alpha4 on leukocytes to form the mucosal addressin molecule LPAM-1, and mice deficient in beta7 show hypoplasia of gut lymphoid tissue due to reduced homing of leukocytes to colon and small bowel (Wagner et al., 1996). Consistently, we observed a reduction mainly in the absolute numbers but not proportions of most immune cells, especially in IFNγ-producing T cell subsets in the lamina propria of colons and small bowels of Beta7^{null} mice after 12 weeks of HFD (FIG. 2A and FIG. 8A). There were no differences in the relative proportions of these subsets in the spleen (FIG. 8B), suggesting that the lack of beta7 integrin does not attenuate systemic immunity. In terms of metabolic parameters, there were no differences in weight gain during 12 weeks of HFD feeding between WT and Beta7^{null} mice (FIG. 2B). Interestingly, HFD-fed Beta7^{null} mice demonstrated improved fasting glucose, glucose tolerance (using glucose tolerance test, GTT), and insulin sensitivity (using insulin tolerance test, ITT) compared to WT mice after 12 weeks of HFD (FIG. 2C). These mice also showed similar food intake, oxygen consumption and carbon dioxide production (FIG. 2D). Histological analysis of bowels in HFD-fed Beta7^{null} mice did not show signs of active colitis. Interestingly, HFD-fed Beta7^{null} mice presented no difference in adipocyte size, but showed a marked reduction in crown-like structures (CLS) in the VAT, along with reduced liver steatosis (FIG. 2E). Consistent with the reduced CLS, HFD Beta7^{null} mice had overall less VAT immune cell infiltrates (FIG. 8E-J). This change was likely not due to the beta7 integrin deficiency imparting an intrinsic defect on T cells to home to VAT, as Beta7^{null} T cells were equally capable at trafficking and engrafting to VAT upon transfer as their wild-type counterparts (FIG. 8K). When Beta7^{null} mice were fed a NCD, we saw little differences in body weight (FIG. 8L), fasting glucose, or insulin tolerance, but some mild improvements in glucose tolerance (FIG. 8M), suggesting that functional glucose modulation by the gut immune system is more pronounced in the setting of HFD but may also be relevant to a lesser degree under normal physiological conditions such as a NCD. Collectively, these results demonstrate that changes to the makeup of the gut immune system may have ramifications in the development of obesity-associated IR.

### Example 5

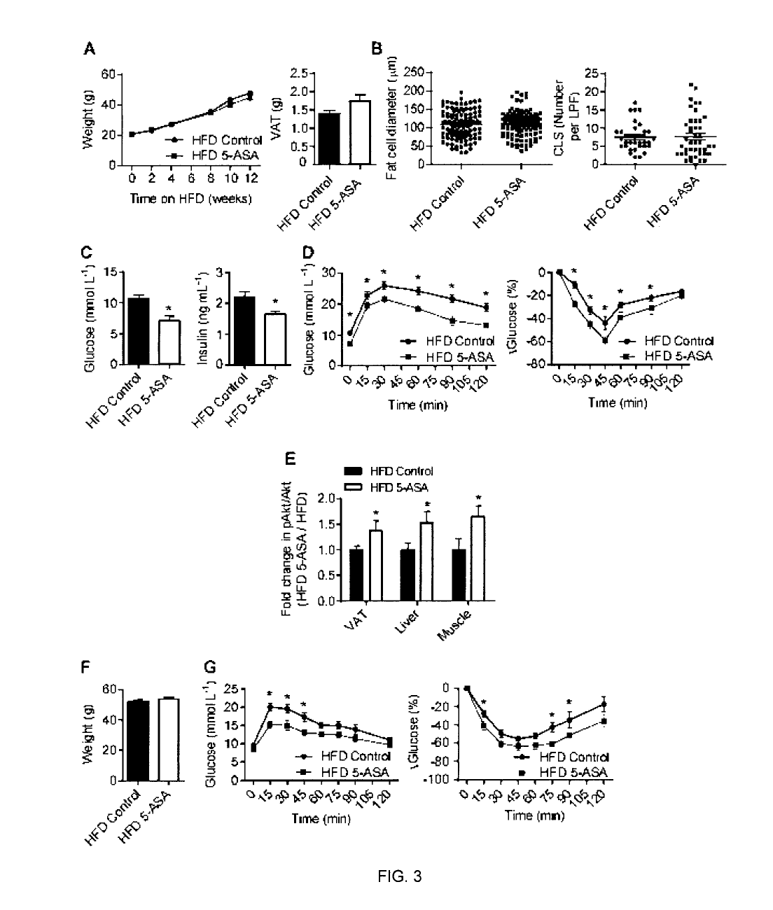

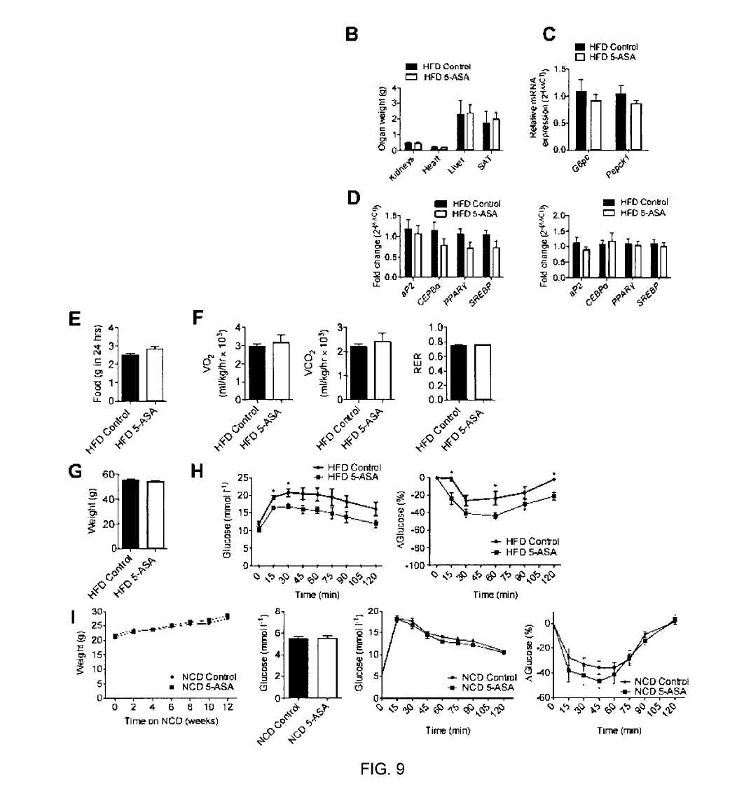

Since obesity is associated with a pro-inflammatory shift in gut immune populations, and the presence of a gut immune system is important in the development of disease, we reasoned that gut-specific anti-inflammatory agent therapies aimed at targeting gut inflammation such as mesalamine (5-ASA), and analogues and derivatives thereof, and/or gut-specific and/or locally gut active PPAR gamma analogues, may have a role in the treatment of metabolic disease. We first fed mice beginning at 6 weeks of age with either HFD or HFD incorporated with 5-ASA (1500 mg/kg/day). After 12-14 weeks of HFD, there was no significant difference in body weight (FIG. 3A, left), VAT weight (FIG. 3A, right), adipocyte size (FIG. 3B, left), number of crown-like structures in VAT (FIG. 3B, right), or organ weights between groups (FIG. 9B). Although we did observe reduced liver steatosis in the 5-ASA treated group, we could not detect significant changes in gluconeogenesis enzyme gene expression, in spite of trends to lower expression with 5-ASA treatment (FIG. 9C). There were also no effects of 5-ASA on expression of adipogenesis-related genes in either VAT or subcutaneous adipose tissue (SAT) (FIG. 9D). Furthermore, there was no difference in food intake, oxygen consumption, carbon dioxide output or respiratory exchange ratio (RER) (FIGS. 9E and 9F). However, mice receiving 5-ASA showed significant improvements in fasting glucose (FIG. 3C, left), fasting insulin (FIG. 3C, right), glucose tolerance (FIG. 3D, left) and insulin tolerance (FIG. 3D, right). Consistent with improved insulin tolerance, 5-ASA treated mice also showed increased phosphorylated-Akt/Akt ratio in VAT, liver and muscle with insulin challenge (FIG. 3E). Similar to the higher dose used, a lower dose (150 mg/kg/day) of 5-ASA also exerted beneficial effects on metabolic disease (FIGS. 9G and H).

### Example 6

We assessed whether 5-ASA, as an example of a gut-specific anti-inflammatory agent, could be used to treat established obesity-associated IR. C57BL/6 mice on HFD for 8 weeks, with established metabolic disease, were switched onto a HFD with 5-ASA for 8 additional weeks and compared to mice on only HFD from the beginning. Similar to the preventative protocol, 5-ASA did not change body weight (FIG. 3F), but did produce significant improvements in glucose tolerance and insulin tolerance (FIG. 3G).

### Example 7

To assess whether the beneficial metabolic effects of 5-ASA require a HFD-induced milieu, we placed 6-week-old C57BL/6 mice on either NCD or NCD with 5-ASA (1500 mg/kg/day). After 12 weeks of treatment, there was little or no difference in body weight, fasting glucose, glucose tolerance, or IR (FIG. 9I). These results suggest that the use of 5-ASA has specific therapeutic effects on glucose homeostasis in the setting of diet-induced obesity.

### Example 8

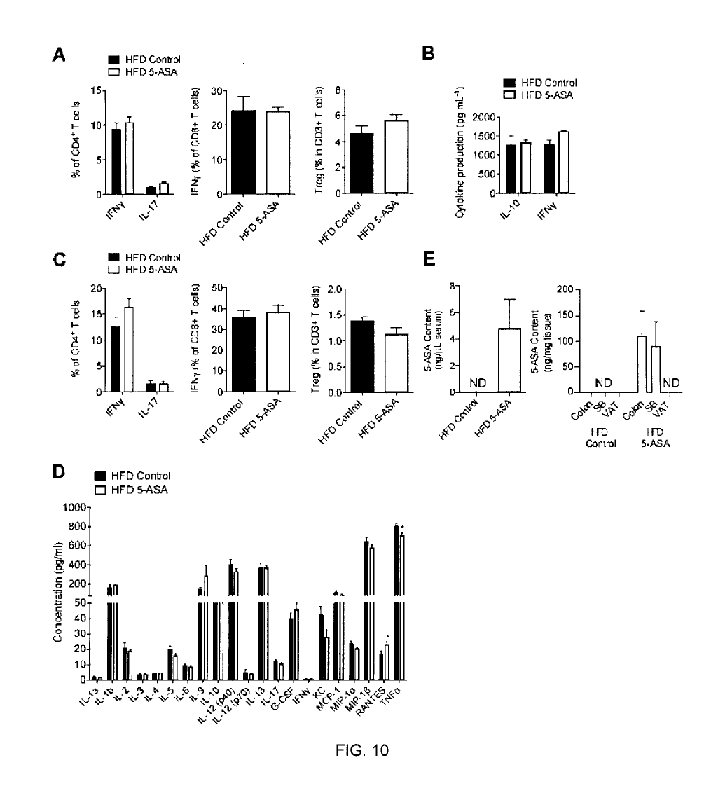

To begin understanding the mechanisms by which 5-ASA can exert effects on glucose homeostasis, we next examined the effects of 5-ASA on systemic and local immune function during HFD feeding. 5-ASA treatment showed no effects on immune cell populations in the spleen (FIG. 10A), on stimulated spleen immune cell cytokine secretion, or on circulating immune cell polarity in the blood (FIGS. 10B and 10C). Similarly, serum levels of cytokines in mice treated with 5-ASA were mostly unchanged, though we did identify a significant but small increase in RANTES and a reduction in TNFα (FIG. 10D). Consistent with little systemic effects on immune cell function, we could identify only traces of 5-ASA compound in the serum of mice, including mice treated with high dose 5-ASA for 12 weeks, by use of high performance liquid chromatography (HPLC) with an internal 4-ASA standard (FIG. 10E, left). Instead, 5-ASA was concentrated in the colon and small bowel (approximately 20× enriched compared to serum, given the density of tissue) and importantly, 5-ASA was undetectable in VAT (FIG. 10E, right). Consistently, as mentioned previously, 5-ASA did not alter expression of adipogenesis-related genes in VAT or SAT (refer back to FIG. 9D). The results are in agreement with previous literature demonstrating poor systemic absorption of 5-ASA upon oral administration (Rousseaux et al., 2005), and highlight the relative specificity of our gut anti-inflammatory therapy.

### Example 9

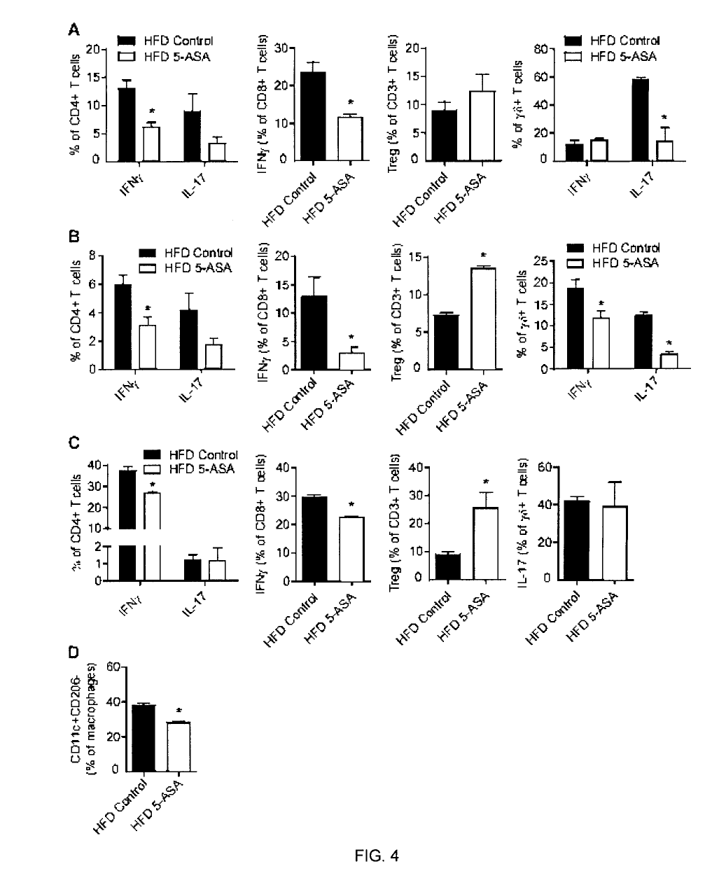

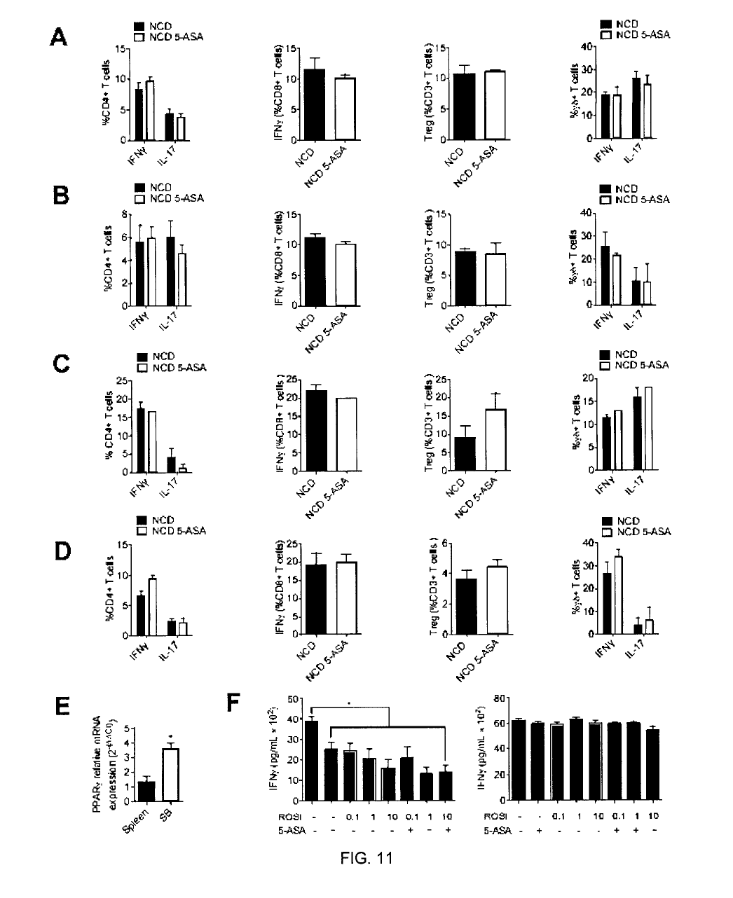

Consistent with a dominant anti-inflammatory effect in the gut, 5-ASA treatment showed an overall reversal of the local pro-inflammatory immune shift in both the colon (FIG. 4A) and small bowel (FIG. 4B), characterized by a reduction in Th1 cells, IFNγ-secreting CD8+ T cells, and IL-17-secreting γδ T cells. There was also a significant increase in Tregs in the small bowel (FIG. 4B). Interestingly, associated with the anti-inflammatory changes in the bowel, 5-ASA also reversed local VAT inflammation by reducing percentages of Th1 cells, IFNγ-secreting CD8+ T cells (FIG. 4C), and M1 inflammatory macrophages (FIG. 4D) in VAT while increasing Tregs (FIG. 4C, third from left). Significant anti-inflammatory effects on immune cell populations were not seen in the bowels or VATs of NCD 5-ASA treated mice compared to untreated NCD mice, suggesting that an increased inflammatory environment was needed to elicit significant differences in immune cell populations (FIG. 11A-D). In line with the anti-inflammatory changes in gut immune populations seen with HFD 5-ASA-fed mice, HFD 5-ASA treatment was also associated with shifts in gut bacteria that are typically seen with administration of the drug, including increased bacterial diversity, increased Firmicutes and increased Clostridiales.

### Example 10

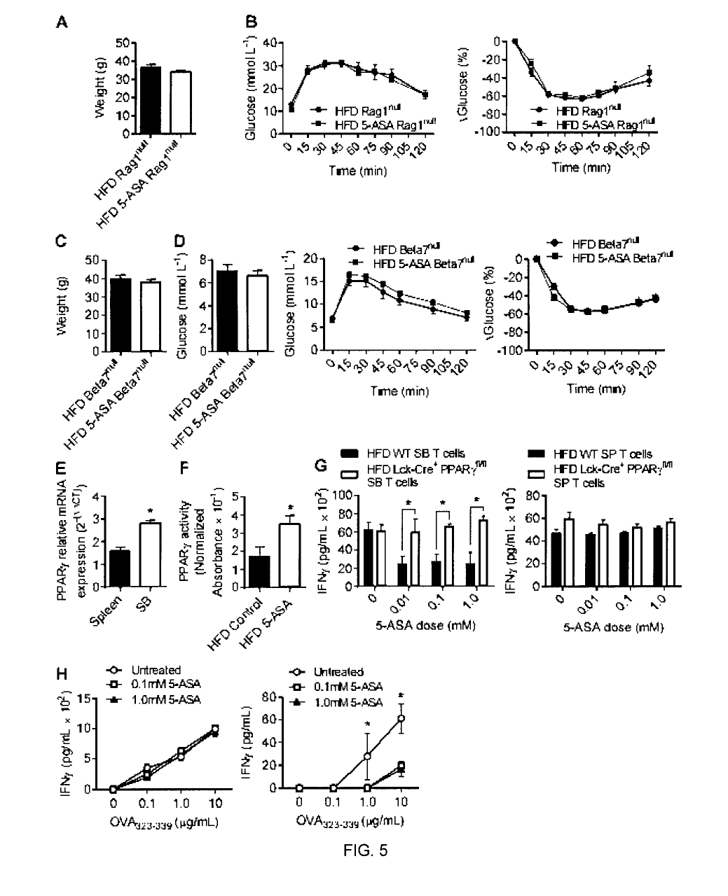

To determine if the effects of 5-ASA were mediated through anti-inflammatory actions that require adaptive immune cells rather than direct effects on gut epithelium, we treated 6-week-old Rag1^{null} mice with HFD 5-ASA. Preventative treatment of Rag1^{null} mice with HFD 5-ASA had no effect on body weight, glucose tolerance or IR (FIGS. 5A and B), suggesting that the beneficial effects of 5-ASA required components of the adaptive immune system. To further pinpoint the location of 5-ASA action on glucose tolerance, we fed Beta7^{null} mice a HFD with 5-ASA. Interestingly, similar to the Rag1^{null} mice, treatment with 5-ASA had no major effects on glucose tolerance and IR (FIGS. 5C and D). Thus, the beneficial metabolic effects of 5-ASA require an “intact” gut immune system.

### Example 11

Since knock-out studies linked potential effects of 5-ASA on glucose metabolism to the gut immune system, and 5-ASA has been reported to possess PPARγ agonist properties (Rousseaux et al., 2005), we next determined if 5-ASA could be directly influencing intestinal immune cell function in HFD through targeting PPARγ. As the effects of 5-ASA were more robust with small bowel T cells than colonic T cells, we focused our studies on small bowel T cells. Indeed, we observed significantly higher PPARγ gene expression in small bowel T cells compared to total splenic T cells in both HFD and NCD-fed mice (FIG. 5E, 11E). Mice fed HFD 5-ASA showed increased PPARγ functional activity in purified small bowel T cells compared to those fed with control HFD (FIG. 5F). We next tested if 5-ASA can suppress IFNγ production in vitro. Indeed, similar to another PPARγ agonist, rosiglitazone, 5-ASA significantly reduced IFNγ production by anti-CD3/CD28-activated small bowel but not splenic T cells (FIG. 11F). Furthermore, loss of PPARγ in T cells (Lck-Cre PPARγfl/fl) abrogated the suppressive effects of 5-ASA, confirming that 5-ASA acts in a PPARγ-dependent manner (FIG. 5G). In addition, 5-ASA indirectly reduced T cell IFNγ expression by modulating intestinal dendritic cell function as shown by reduced IFNγ levels in antigen-specific co-culture systems using OT-II CD4+ T cells and 5-ASA pre-treated small bowel but not splenic dendritic cells (FIG. 5H).

### Example 12

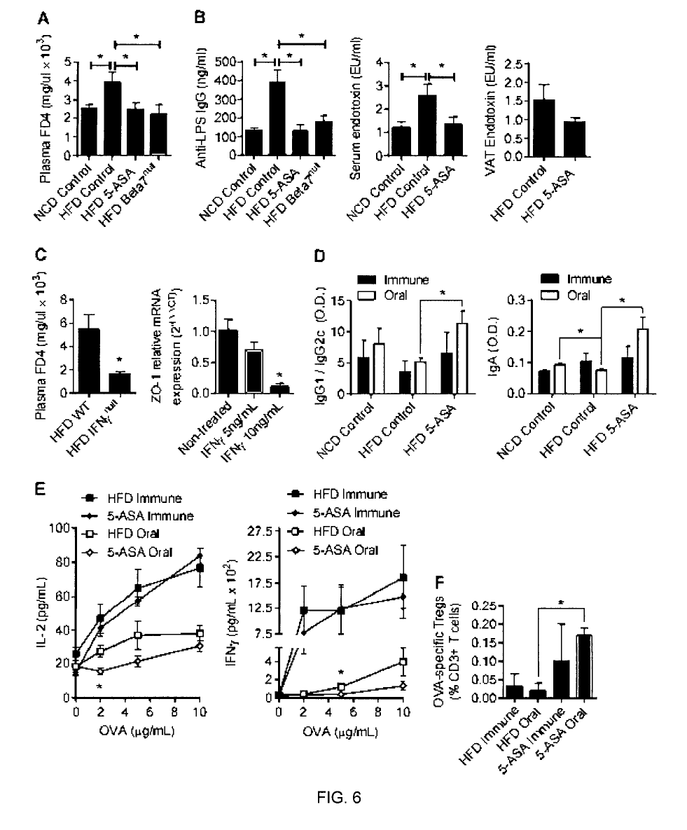

Since both diet-induced obesity and intestinal inflammation are associated with impairment of the gut epithelial barrier, which can trigger systemic endotoxemia and IR (Cani et al., 2007; Wang et al., 2014), we next investigated the effects of 5-ASA on intestinal permeability, and serum and VAT endotoxin levels. 5-ASA treatment induced significant improvements in intestinal epithelial barrier permeability, as measured by fluorescence FD4 assay (FIG. 6A), which is consistent with the reduced gut immune cell inflammatory shift and the overall improvement in glucose homeostasis observed previously. Moreover, IgG responses to LPS were markedly diminished in 5-ASA treated mice (FIG. 6B, left) accompanied by reduced levels of serum endotoxin (FIG. 6B, middle). VAT endotoxin levels also trended lower in 5-ASA-treated mice, though this result did not reach significance (FIG. 6B, right). Taken together, these results show effects of 5-ASA on reducing HFD-induced gut leakage to endotoxins.

### Example 13

Because HFD 5-ASA reduces IFNγ expression compared to control HFD, and is associated with improvements in intestinal permeability, we next assessed the role of IFNγ in intestinal permeability during HFD feeding. HFD-fed IFNγ-deficient mice showed improved intestinal barrier function reflected in reduced plasma FD4 levels (FIG. 6C, left). IFNγ was also able to reduce ZO-1 tight junction gene expression in intestinal epithelial cells, suggesting one possible mechanism for its ability to influence gut permeability (FIG. 6C, right). Thus, the shifts in HFD bowel cells to IFNγ-producing cells likely impact metabolic function at the level of intestinal permeability. To further corroborate this notion, we assessed bowel permeability in Beta7^{null} mice, which showed reduced numbers of intestinal immune cells, that most prominently affected IFNγ-expressing T cells. Beta7^{null} mice showed improved/reduced intestinal permeability as measured by FD4 assay and reduced anti-LPS IgG (FIGS. 6A and B, left).

### Example 14

While permeability-related gut-derived endotoxin alone may contribute to VAT inflammation and potentially IR (Caesar et al., 2012), it is thought that this trigger works alongside other gut-associated antigens to activate antigen-specific T cells in VAT, thereby influencing glucose homeostasis (Wang et al., 2010). Thus, to further understand how a gut-specific anti-inflammatory agent may contribute to reduced inflammation in VAT, we examined the effects of 5-ASA on oral immune tolerance to gut-derived antigen. NCD, HFD, or HFD 5-ASA-fed C57BL/6 mice were administered oral ovalbumin (OVA) antigen for 1 week prior to immunization with OVA-CFA. Interestingly, HFD 5-ASA-fed mice showed a stronger oral tolerance response to OVA antigen systemically, as reflected by an increased OVA-specific IgG1/IgG2c ratio (indicative of reduced Th1 inflammatory responses), and a nearly threefold increase in OVA-specific IgA (FIG. 6D). Moreover, draining lymph nodes in mice fed HFD 5-ASA demonstrated a reduction in OVA-specific T cell-derived IL-2 and IFNγ, which is also consistent with the improved oral tolerance and reduced antigen-specific inflammation to gut antigen (FIG. 6E). Finally, 5-ASA treatment induced a nearly fourfold increase in antigen-specific Tregs to OVA in VAT as measured using OVA/I-A^{b} tetramers (FIG. 6F). Collectively, the data suggest that reducing low-grade inflammation in the gut during HFD feeding can impact multiple pathways associated with IR, including gut barrier function, tolerance to gut-derived antigen, and antigen-specific immunity to gut-derived antigen in VAT. Taken together, these results suggest that anti-inflammatory targeting of gut immune cells is a novel approach to treat obesity-related IR.

### Example 15

Sulfasalazine (tablet, 2000-4000 mg per day) is a prodrug that contains mesalamine bound to the antibiotic sulfapyridine via an azo bond that is cleaved by (colonic) bacteria to free up the active mesalamine. This formulation reduces the absorption in the small bowel and localizes the absorption more in the colon (and terminal ileum) (approximately 20% is absorbed in small bowel, the remaining has local effects in the colon). Sulfasalazine powder is incorporated directly into the mouse HFD at between 200 mg/kg/day and 1600 mg/kg/day, corresponding to the equivalent human dosage of 1000-8000 mg/day.

### Example 16

Asacol (tablet, 400-600 mg per day) is formed by coating mesalamine with a pH sensitive coating (dibutyl phthalate). The coating dissolves when the pH is greater than 7, which typically first occurs in the terminal ileum, and therefore the majority of the drug is locally active in the terminal ileum and colon. Asacol tablet is crushed and incorporated directly into the mouse HFD at between 80 mg/kg/day and 250 mg/kg/day, corresponding to the equivalent human dosage of 200-1200 mg/day.

### Example 17

Delzicol (capsule, 2400 mg per day) is formed by coating mesalamine with a pH sensitive coating (dibutyl sebacate) and is a delayed release that is most active in terminal ileum and colon. Delzicol powder is incorporated directly into the mouse HFD at between 250 mg/kg/day and 1000 mg/kg/day, corresponding to the equivalent human dosage of 1200-4800 mg/day.

### Example 18

Pentasa (capsule, 3000-4000 mg per day) is mesalamine in coated permeable microgranules, which causes a slow and even release of mesalamine throughout the small bowel and colon. Pentasa is generally taken 3-4 times per day. Pentasa powder is incorporated directly into the mouse HFD at between 300 mg/kg/day and 1600 mg/kg/day, corresponding to the equivalent human dosage of 1500-8000 mg/day.

### Example 19

Lialda (tablet, 2400-4800 mg once a day) is a very slow release mesalamine given only once a day. Lialda tablets is crushed and incorporated directly in the mouse HFD at between 250 mg/kg/day and 2000 mg/kg/day, corresponding to the equivalent human dosage of 1200-9600 mg/day.

### Example 20

Apriso (capsule, 1500 mg once a day) is a very slow release mesalamine given only once a day. Apriso powder is incorporated directly into the mouse HFD at between 150 mg/kg/day and 620 mg/kg/day, corresponding to the equivalent human dosage of 750-3000 mg/day.

### Example 21

Olsalazine (capsule, 500-1000 mg once a day) releases mesalamine in the large intestine. Olsalazine powder is incorporated directly into the mouse HFD at between 50 mg/kg/day and 400 mg/kg/day, corresponding to the equivalent human dosage of 250-2000 mg/day.

### Example 22

Balsalazide (capsule, 3 times 750 mg three times a day (6750 mg per day) releases mesalamine in the large intestine. Balsalazide powder is incorporated directly into the mouse HFD at between 700 mg/kg/day and 2800 mg/kg/day, corresponding to the equivalent human dosage of 3400-13500 mg/day.

### Example 23

GED-0507-34 is a PPARgamma modulator and has been assessed in clinical trials in prolonged-release tablets. GED-0507-34 powder is incorporated directly into the mouse HFD at between 10 mg/kg/day and 80 mg/kg/day, corresponding to the equivalent human dosage of 40-400 mg/day.

### Example 24

5-ASA patients are selected who have high glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of mesalamine. Mesalamine is administered in a dose of between about 720 and 7200 mg/per day and patients are monitored for improvement of high glucose levels.

### Example 25

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of sulfasalazine. Sulfasalazine is administered in a dose of between about 1000 and 8000 mg/per day and patients are monitored for improvement of high glucose levels.

### Example 26

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of asacol. Asacol is administered in a dose of between about 200 and 1200 mg/per day and patients are monitored for improvement of high glucose levels.

### Example 27

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of delzicol. Delzicol is administered in a dose of between about 1200 and 4800 mg/per day and patients are monitored for improvement of high glucose levels.

### Example 28—Use of Gut-Specific Anti-Inflammatories to Treat High Glucose Levels

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of pentasa. Pentasa is administered in a dose of between about 1500 and 8000 mg/per day and patients are monitored for improvement of high glucose levels.

### Example 29

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of lialda. Lialda is administered in a dose of between about 1200 and 9600 mg/per day and patients are monitored for improvement of high glucose levels.

### Example 30

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of apriso. Apriso is administered in a dose of between about 750 and 3000 mg/per day and patients are monitored for improvement of clinical manifestation (a).

### Example 31

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of olsalazine. Olsalazine is administered in a dose of between about 250 and 2000 mg/per day and patients are monitored for improvement of high glucose levels.

### Example 32

Patients are selected who have high blood glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of balsalazide. Balsalazide is administered in a dose of between about 3400 and 13500 mg/per day and patients are monitored for improvement of high glucose levels.

### Example 33

Patients are selected who have high glucose levels, as can be determined using e.g. one of fasting blood glucose, oral glucose tolerance test, and/or the haemoglobin A1C test, that may be a result of obesity, type 1 diabetes and/or type 2 diabetes and are in need of treatment. Selected patients are administered a pharmaceutically acceptable formulation of GED-0507-34. GED-0507-34 is administered in a dose of between about 40 and 400 mg/per day and patients are monitored for improvement of high glucose levels.

The above-described embodiments are intended to be examples only. Alterations, modifications and variations can be effected to the particular embodiments by those of skill in the art without departing from the scope, which is defined solely by the claims appended hereto.

### DISCUSSION

We have identified the gut immune system as an active orchestrator and therapeutic target in obesity-related IR. Previous work has shown that HFD increases ileal TNFα mRNA, induces expression of TLR4 and NF-κB in small bowels (Ding et al., 2010; Wang et al., 2013) and also increases IL-1β, IL-12p40, NF-κB, and TLR4 in colons of DIO mice (Kim et al., 2012; Li et al., 2008). Consistently, we show that diet-induced obesity promotes a pro-inflammatory shift in gut immune cell populations, characterized by reduced lamina propria Foxp3+ Treg cells, increased IFNγ-producing Th1 and CD8+ T cells, as well as increased IL-17-producing γδ T cells. Similar to the changes in mice, altered ratios of Tbet+ cells:Foxp3+ Treg cells, as well as changes in CD8+ T cells, were found in both small and large bowels of obese humans, though these studies involved the use of negative margin specimens from patients with tumors, and thus need more rigorous follow-up in additional cohorts of patients, including bariatric patients. A recent report has also demonstrated reduction in IL-22 in the gut of obese mice post immune challenge (Wang et al., 2014). Consistently, we saw reduced percentages of NKp46+CD4− ILCs, which are important producers of IL-22. Moreover, the pro-inflammatory shift in immune cell populations observed in the gut was not associated with obvious inflammatory histological changes, and so we classify this pro-inflammatory shift as a sub-histological change or “low-grade subclinical inflammation”.

We next investigated if the gut immune system as a whole could exert systemic effects on glucose homeostasis. In this model, we utilized Beta7^{null} mice, which have marked hypoplasia of the gut lymphoid system. We noted improved metabolic parameters in the Beta7^{null} mice despite similar body weights. These mice showed reduced immune cell infiltrates in the gut during HFD, including reductions in IFNγ-producing CD4+ and CD8+ T cells, consistent with a potential pathogenic role for some intestinal immune cells in diet-induced obesity. However, additional work is needed to rule out whether other off-target effects of this molecule, such as potential traffic to other tissues, exist in the setting of diet-induced obesity which might also contribute to the phenotype. Furthermore, Beta7^{null} mice are susceptible to bacterial overgrowth (Wagner et al., 1996) which can cause changes in the microbiome and contribute to the observed phenotype; this phenotype may be similar to the recently described phenotype in lymphotoxin-deficient mice that show hypoplasia of Peyer's patches and improved glucose tolerance due to altered colonization of segmented filamentous bacteria (SFB) and reduced energy harvesting bacteria in the gut (Upadhyay et al., 2012). Nonetheless, taking phenotypic data between both models, it appears that some level of active gut inflammation contributes to downstream pathways, ultimately leading to obesity or related IR. Potential pathways include modulation of the gut flora with effects on energy harvesting bacteria (Upadhyay et al., 2012), bile acid and short chain fatty acid release (Brown et al., 2013), modulation of the gut epithelial barrier (Pastorelli et al., 2013), control of gut hormone release such as GLP-1 leading to hyperinsulinemia (Kahles et al., 2014), and a role in dictating inflammatory responses to gut-derived antigen and endotoxin (Caesar et al., 2012; Wang et al., 2010).

In our cohorts of HFD Beta7^{null} mice, we observed an overall improvement in gut barrier function, characterized by reduced FD4 and anti-LPS response; these findings are potentially linked to reduced infiltrates of IFNγ-producing cells in the bowel, as IFNγ has direct pathological effects on disrupting barrier function (Beaurepaire et al., 2009). Consistently, we observed improved barrier function in HFD IFNγ knockout mice compared to HFD controls, implicating local intestinal IFNγ production as one critical pathogenic mediator on intestinal permeability in the setting of diet-induced obesity.

Indeed, the overall HFD-induced phenotype of intestinal immune cells observed in WT mice is consistent with changes described in other diseases characterized by breech of intestinal barrier, dysbiosis, and subsequent anti-bacterial immune response (Petnicki-Ocwieja et al., 2009). Lamina propria CD4+Foxp3+ Tregs, in particular, are critical in maintaining a tolerant response to gut microbiota, and are reduced in the presence of intestinal barrier defects. In healthy hosts, Tregs maintain the intestinal barrier through promotion of TGF-β-dependent microbiota-specific IgA responses (Cong et al., 2009). Upon breech of the barrier, Tregs are required to suppress Th1 responses via IL-10 and TGF-β (Cong et al., 2009). Our observed HFD-associated reductions in lamina propria Tregs, and increases in pro-inflammatory IFNγ-secreting Th1 and CD8+ cells, as well as IL-17-producing γδ T cells are thus consistent immunologically with intestinal barrier breech. Several studies have shown that diet-induced obesity is also associated with a breech in the intestinal barrier, leading to increases in circulating levels of gut-derived microbial products, such as LPS (Cani et al., 2007; Cani et al., 2008). In addition to direct leakage, gut-derived LPS can be transported along with chylomicrons into circulation (Ghoshal et al., 2009). While we described one mechanism of immune cell IFNγ-mediated effect on the intestinal barrier during diet-induced obesity, it is also possible that changes in IL-10, which would accompany reductions in Tregs, or changes in the inflammatory status of the intestinal epithelial cells actively contribute to decreased barrier function in obesity. Indeed, IL-10 was shown to promote intestinal barrier mucin production (Hasnain et al., 2013), while a recent study showed improvements in intestinal barrier function in HFD mice lacking the pro-inflammatory molecule, MyD88, only in intestinal epithelial cells (Everard et al., 2014). In the latter study, there was also an improvement in glucose homeostasis associated with knockdown of intestinal epithelial cell MyD88, in agreement with our data showing an overall pathogenic role for intestinal inflammation in diet-induced obesity related metabolic disease. Thus, a combination of cues from both cells of the intestinal immune system, as we have described, as well as innate pathways within the intestinal epithelium, collaborate to regulate intestinal barrier function and downstream glucose homeostasis during diet-induced obesity.

We further show that inhibition of low-grade gut inflammation with the local gut-specific anti-inflammatory agent, 5-ASA, during HFD feeding can alter systemic glucose metabolism. Treatment with gut anti-inflammatory agents, including 5-ASA and Balsalazide, has beneficial effects on the intactness of the gut epithelial barrier in models of IBD (Di Paolo et al., 1996; Liu et al., 2009), and we show similar beneficial effects on gut barrier functions during HFD feeding. These beneficial effects are linked to reduced levels of inflammatory cytokines, such as TNFα and IFNγ, which can directly worsen gut bacteria leakage through the barrier (Barreau et al., 2010; Beaurepaire et al., 2009). Accordingly, we show similar alterations in intestinal IFNγ-producing cells contribute to gut barrier defects in the setting of diet-induced obesity. In addition to its well-described role as a COX-2 inhibitor, 5-ASA has PPARγ agonistic effects, which may also contribute to our observed anti-inflammatory phenotype (Rousseaux et al., 2005). We noted increased PPARγ activity from bowel T cells of HFD 5-ASA-fed mice, and that PPARγ contributes to 5-ASA inhibitory effects on IFNγ production by intestinal T cells in vitro. Interestingly, PPARγ induction in T cells can also bolster Treg function and numbers in other tissues, including VAT (Cipolletta et al., 2012). However, systemic effects of PPARγ agonism in fat or liver are unlikely in our study due to minimal metabolic effects seen in 5-ASA-fed Rag1^{null} mice and Beta7^{null} mice, the lack of changes in expression of key adipogenesis genes in both VAT and SAT, and the lack of detectable compound in VAT of HFD 5-ASA-fed mice. Thus, intestinal immune cell PPARγ may be another potential target of action for immune modulatory drugs with PPARγ agonistic effects.

Consistent with other reports (Andrews et al., 2011), we also noted that 5-ASA could elicit changes in the gut bacteria, including increased bacterial diversity, and increased abundances of Firmicutes, Clostridiales, and Ruminococcaceae. However, while these changes could reflect primary effects of the drug, they could also be secondary to reduced inflammation (Andrews et al., 2011; Sartor, 2010). Reduced bacterial diversity, as well as decreases in certain Clostridial groups and Ruminococcaceae have been linked to increased inflammation in IBD (Sartor, 2010). Indeed, Ruminococcaceae are prominent producers of short-chain fatty acids, including butyrate, which have protective activity in the intestine (Sartor, 2010). Thus, it will be an interesting future direction to tease out specific effects of 5-ASA associated microbial influences on facilitating improvements in metabolic syndrome.

We were able to obtain beneficial effects on glucose tolerance using a 5-ASA dose range of 150 mg/kg/day up to 1500 mg/kg/day in mice, which using body surface area calculations (Reagan-Shaw et al., 2008) equates to approximate equivalent doses of 730 mg/day up to 7 g/day in a 60 kg human. Typical daily maintenance dosing of 5-ASA for mild to moderate IBD is varied but often ranges between 1.5-4.8 g/day (Burger and Travis, 2011). Thus, our work highlights novel uses of such drugs, e.g. mesalamine (5-aminosalicylic acid), along with various analogues and variants including sulfasalazine, asacol, delzicol, pentasa, lialda, apriso, olsalazine, balsalazide and GED-0507-34 and pharmaceutically acceptable salts, solvates, or esters of any of the foregoing, in treating high blood glucose levels, and/or glucose intolerance and/or resulting from e.g. Type 1 diabetes, Type 2 diabetes and/or obesity.

Because the improvements in systemic glucose tolerance with 5-ASA treatment were found to be dependent on adaptive and gut immune systems, this notion suggests a critical role for controlling T or B cell-mediated gut inflammation in governing glucose homeostasis. The observed direct effects of 5-ASA in vitro on purified intestinal dendritic cells in modulating antigen-specific T cell responses and ensuing IFNγ production also highlight potential cross-talks between intestinal adaptive and innate immune cells in mediating the effects of 5-ASA. The improvements in both intestinal and VAT inflammation in both 5-ASA-treated or Beta7^{null} HFD-fed mice, without affecting the overall inflammatory status in systemic hematolymphoid organs such as the spleen, suggest a possible linked circuit between adipose tissue and bowel inflammation. Consistent with this concept, other studies suggest that bowel inflammation might directly contribute to VAT inflammation (Li et al., 2008; Teixeira et al., 2011). For instance, induction of colitis during HFD leads to marked increases in VAT macrophages, lymphocytes and neutrophils (Teixeira et al., 2011). Such results raise the possibility of downstream trafficking between immune cells of the bowel and VAT, or that tolerance to leaked gut soluble antigens in VAT is dependent on mechanisms governed by the gut immune system. Additional studies are needed to determine whether bowel immune cells routinely traffic to VAT and whether trafficking of gut-derived anti-inflammatory immune cells (or reduced trafficking of gut inflammatory cells) to VAT represents another mechanism of action of 5-ASA.

Another contributing role of the gut immune system during HFD may be in dictating downstream systemic inflammation to soluble gut-derived antigens, including in metabolic tissues like VAT, where inflammation directly impacts systemic disease. The improved oral tolerance may also manifest as reduced inflammatory responses, including IgG against gut-derived endotoxin. Oral tolerance to gut-derived antigens has been linked to reduced inflammation in VAT and improvements in IR in previous reports, though the mechanisms behind this observation were unknown (Wang et al., 2010). We show that aberrant handling of gut antigen is likely due to the inflammatory environment in the gut during HFD, which is reversible with gut anti-inflammatory medication. This HFD-induced low-grade inflammation may be a key trigger that initiates antigen-specific T cell responses in VAT, linking the inflammatory phenotype we describe in the bowel to downstream responses in VAT.

Overall, our work shows that low-grade inflammation in gut immune cells is a functional alteration induced by HFD with implications in IR. Reducing low-grade gut inflammation also leads to reduction in VAT inflammation and improvements in metabolic homeostasis. These effects are dependent on the adaptive immune system and gut immunity. Thus, compounds that locally reduce gut inflammation may represent a novel approach in the control of obesity-related IR.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCES

Andrews, C. N., Griffiths, T. A., Kaufman, J., Vergnolle, N., Surette, M. G., and Rioux, K. P. (2011). Mesalazine (5-aminosalicylic acid) alters faecal bacterial profiles, but not mucosal proteolytic activity in diarrhoea-predominant irritable bowel syndrome. Alimentary pharmacology & therapeutics 34, 374-383.
Backhed, F., Ding, H., Wang, T., Hooper, L. V., Koh, G. Y., Nagy, A., Semenkovich, C. F., and Gordon, J. I. (2004). The gut microbiota as an environmental factor that regulates fat storage. Proceedings of the National Academy of Sciences of the United States of America 101, 15718-15723.
Backhed, F., Manchester, J. K., Semenkovich, C. F., and Gordon, J. I. (2007). Mechanisms underlying the resistance to diet-induced obesity in germ-free mice. Proceedings of the National Academy of Sciences of the United States of America 104, 979-984.
Barreau, F., Madre, C., Meinzer, U., Berrebi, D., Dussaillant, M., Merlin, F., Eckmann, L., Karin, M., Sterkers, G., Bonacorsi, S., et al. (2010). Nod2 regulates the host response towards microflora by modulating T cell function and epithelial permeability in mouse Peyer's patches. Gut 59, 207-217.
Beaurepaire, C., Smyth, D., and McKay, D. M. (2009). Interferon-gamma regulation of intestinal epithelial permeability. Journal of interferon & cytokine research: the official journal of the International Society for Interferon and Cytokine Research 29, 133-144.
Bernink, J. H., Peters, C. P., Munneke, M., to Velde, A. A., Meijer, S. L., Weijer, K., Hreggvidsdottir, H. S., Heinsbroek, S. E., Legrand, N., Buskens, C. J., et al. (2013). Human type 1 innate lymphoid cells accumulate in inflamed mucosal tissues. Nature immunology 14, 221-229.
Bokulich, N. A., Subramanian, S., Faith, J. J., Gevers, D., Gordon, J. I., Knight, R., Mills, D. A., and Caporaso, J. G. (2013). Quality-filtering vastly improves diversity estimates from Illumina amplicon sequencing. Nature Methods 10, 57-59.
Brown, E. M., Sadarangani, M., and Finlay, B. B. (2013). The role of the immune system in governing host-microbe interactions in the intestine. Nature immunology 14, 660-667.
Burger, D., and Travis, S. (2011). Conventional medical management of inflammatory bowel disease. Gastroenterology 140, 1827-1837 e1822.
Caesar, R., Reigstad, C. S., Backhed, H. K., Reinhardt, C., Ketonen, M., Lunden, G. O., Cani, P. D., and Backhed, F. (2012). Gut-derived lipopolysaccharide augments adipose macrophage accumulation but is not essential for impaired glucose or insulin tolerance in mice. Gut 61, 1701-1707.
Cani, P. D., Amar, J., Iglesias, M. A., Poggi, M., Knauf, C., Bastelica, D., Neyrinck, A. M., Fava, F., Tuohy, K. M., Chabo, C., et al. (2007). Metabolic endotoxemia initiates obesity and insulin resistance. Diabetes 56, 1761-1772.
Cani, P. D., Bibiloni, R., Knauf, C., Waget, A., Neyrinck, A. M., Delzenne, N. M., and Burcelin, R. (2008). Changes in gut microbiota control metabolic endotoxemia-induced inflammation in high-fat diet-induced obesity and diabetes in mice. Diabetes 57, 1470-1481.
Caporaso, J. G., Bittinger, K., Bushman, F. D., DeSantis, T. Z., Andersen, G. L., and Knight, R. (2010). PyNAST: a flexible tool for aligning sequences to a template alignment. Bioinformatics 26, 266-267.
Caporaso, J. G., Lauber, C. L., Walters, W. A., Berg-Lyons, D., Huntley, J., Fierer, N., Owens, S. M., Betley, J., Fraser, L., Bauer, M., Gormley, N., Gilbert, J. A., Smith, G., Knight, R. (2012). Ultra high-throughout microbial community analysis on the IIlumina HiSeq and MiSeq platforms. ISME Journal 6, 1621-1621.
Caporaso, G., Kuczynski, J., Stombaugh, J., Bittinger, K., Bushman, F. D., Costello, E. K., Fierer, N., Pena, A. G., Goodrich, J. K., Gordon, J. I., Huttley, G. A., Kelley, S. T., Knights, D., Koenig, J. E., Ley, R. E., Lozupone, C. A., McDonald, D., Muegge, B. D., Pirrung, M., Reeder, J., Sevinsky, J. R., Turnbaugh, P. J., Walters, W. A., Widmann, J., Yatsunenko, T., Zaneveld, J., Knight, R. (2010). QIIME allows analysis of high-throughput community sequencing data. Nature Methods 7, 335-336.
Cipolletta, D., Feuerer, M., Li, A., Kamei, N., Lee, J., Shoelson, S. E., Benoist, C., and Mathis, D. (2012). PPAR-gamma is a major driver of the accumulation and phenotype of adipose tissue Treg cells. Nature 486, 549-553.
Cong, Y., Feng, T., Fujihashi, K., Schoeb, T. R., and Elson, C. O. (2009). A dominant, coordinated T regulatory cell-IgA response to the intestinal microbiota. Proceedings of the National Academy of Sciences of the United States of America 106, 19256-19261.
de La Serre, C. B., Ellis, C. L., Lee, J., Hartman, A. L., Rutledge, J. C., and Raybould, H. E. (2010). Propensity to high-fat diet-induced obesity in rats is associated with changes in the gut microbiota and gut inflammation. American journal of physiology. Gastrointestinal and liver physiology 299, G440-448.
DeSantis, T. Z., Hugenholtz, P., Larsen, N., Rojas, M., Brodie, E. L., Keller, K., Huber, T., Dalevi, D., Hu, P., and Andersen, G. L. (2006). Greengenes, a chimera-checked 16S rRNA gene database and workbench compatible with ARB. Applied and Environmental Microbiology 72, 5069-5072.
Di Paolo, M. C., Merrett, M. N., Crotty, B., and Jewell, D. P. (1996). 5-Aminosalicylic acid inhibits the impaired epithelial barrier function induced by gamma interferon. Gut 38, 115-119.
Ding, S., Chi, M. M., Scull, B. P., Rigby, R., Schwerbrock, N. M., Magness, S., Jobin, C., and Lund, P. K. (2010). High-fat diet: bacteria interactions promote intestinal inflammation which precedes and correlates with obesity and insulin resistance in mouse. PloS one 5, e12191.
Dong, C. X., Zhao, W., Solomon, C., Rowland, K. J., Ackerley, C., Robine, S., Holzenberger, M., Gonska, T., and Brubaker, P. L. (2014). The intestinal epithelial insulin-like growth factor-1 receptor links glucagon-like peptide-2 action to gut barrier function. Endocrinology 155, 370-379.
Edgar, R. C. (2010). Search and clustering orders of magnitude faster than BLAST. Bioinformatics 26, 2460-2461.
Everard, A., Geurts, L., Caesar, R., Van Hul, M., Matamoros, S., Duparc, T., Denis, R. G., Cochez, P., Pierard, F., Castel, J., et al. (2014). Intestinal epithelial MyD88 is a sensor switching host metabolism towards obesity according to nutritional status. Nature communications 5, 5648.
Feuerer, M., Herrero, L., Cipolletta, D., Naaz, A., Wong, J., Nayer, A., Lee, J., Goldfine, A. B., Benoist, C., Shoelson, S., et al. (2009). Lean, but not obese, fat is enriched for a unique population of regulatory T cells that affect metabolic parameters. Nature medicine 15, 930-939.
Fritz, J. H., Rojas, O. L., Simard, N., McCarthy, D. D., Hapfelmeier, S., Rubino, S., Robertson, S. J., Larijani, M., Gosselin, J., Ivanov, I I, et al. (2012). Acquisition of a multifunctional IgA+ plasma cell phenotype in the gut. Nature 481, 199-203.
Geddes, K., Rubino, S. J., Magalhaes, J. G., Streutker, C., Le Bourhis, L., Cho, J. H., Robertson, S. J., Kim, C. J., Kaul, R., Philpott, D. J., et al. (2011). Identification of an innate T helper type 17 response to intestinal bacterial pathogens. Nature medicine 17, 837-844.
Ghoshal, S., Witta, J., Zhong, J., de Villiers, W., and Eckhardt, E. (2009). Chylomicrons promote intestinal absorption of lipopolysaccharides. Journal of lipid research 50, 90-97.
Goldfine, A. B., Fonseca, V., Jablonski, K. A., Pyle, L., Staten, M. A., and Shoelson, S. E. (2010). The effects of salsalate on glycemic control in patients with type 2 diabetes: a randomized trial. Annals of internal medicine 152, 346-357.
Gregor, M. F., and Hotamisligil, G. S. (2011). Inflammatory mechanisms in obesity. Annual review of immunology 29, 415-445.
Hasnain, S. Z., Tauro, S., Das, I., Tong, H., Chen, A. C., Jeffery, P. L., McDonald, V., Florin, T. H., and McGuckin, M. A. (2013). IL-10 promotes production of intestinal mucus by suppressing protein misfolding and endoplasmic reticulum stress in goblet cells. Gastroenterology 144, 357-368 e359.
Hong, L., Jiang, W., Zheng, W., Zeng, S. (2011). HPLC analysis of para-aminosalicylic acid and its metabolite in plasma, cerebrospinal fluid and brain tissues. J Pharm Biomed Anal 54, 1101-1109.
Johnson, A. M., and Olefsky, J. M. (2013). The origins and drivers of insulin resistance. Cell 152, 673-684.
Kahles, F., Meyer, C., Mollmann, J., Diebold, S., Findeisen, H. M., Lebherz, C., Trautwein, C., Koch, A., Tacke, F., Marx, N., et al. (2014). GLP-1 Secretion Is Increased by Inflammatory Stimuli in an IL-6-Dependent Manner, Leading to Hyperinsulinemia and Blood Glucose Lowering. Diabetes.
Kim, K. A., Gu, W., Lee, I. A., Joh, E. H., and Kim, D. H. (2012). High fat diet-induced gut microbiota exacerbates inflammation and obesity in mice via the TLR4 signaling pathway. PloS one 7, e47713.
Kirchberger, S., Royston, D. J., Boulard, O., Thornton, E., Franchini, F., Szabady, R. L., Harrison, O., and Powrie, F. (2013). Innate lymphoid cells sustain colon cancer through production of interleukin-22 in a mouse model. The Journal of experimental medicine 210, 917-931.
Larsen, C. M., Faulenbach, M., Vaag, A., Volund, A., Ehses, J. A., Seifert, B., Mandrup-Poulsen, T., and Donath, M. Y. (2007). Interleukin-1-receptor antagonist in type 2 diabetes mellitus. The New England journal of medicine 356, 1517-1526.
Li, H., Lelliott, C., Hakansson, P., Ploj, K., Tuneld, A., Verolin-Johansson, M., Benthem, L., Carlsson, B., Storlien, L., and Michaelsson, E. (2008). Intestinal, adipose, and liver inflammation in diet-induced obese mice. Metabolism: clinical and experimental 57, 1704-1710.
Liu, X. C., Mei, Q., Xu, J. M., and Hu, J. (2009). Balsalazine decreases intestinal mucosal permeability of dextran sulfate sodium-induced colitis in mice. Acta pharmacologica Sinica 30, 987-993.
Lumeng, C. N., Bodzin, J. L., and Saltiel, A. R. (2007). Obesity induces a phenotypic switch in adipose tissue macrophage polarization. The Journal of clinical investigation 117, 175-184.
Masella, A. P., Bartram, A. K., Truszkowski, J. M., Brown, D. G., Neufeld, J. D. (2012). PANDAseq: paired-end assembler for illumina sequences. BMC Bioinformatics 13, 31.
Maughan, H., Wang, P. W., Diaz Caballero, J., Fung, P., Gong, Y., Donaldson, S. L., Yuan, L., Keshavjee, S., Zhang, Y., Yau, Y. C. W., Waters, V. J., Tullis, D. E., Hwang, D. M., and Guttman, D. S. (2012). Analysis of the cystic fibrosis lung microbiota via serial IIlumina sequencing of bacterial 16S rRNA hypervariable regions. PLoS One 7, e45791.
Membrez, M., Blancher, F., Jaquet, M., Bibiloni, R., Cani, P. D., Burcelin, R. G., Corthesy, I., Mace, K., and Chou, C. J. (2008). Gut microbiota modulation with norfloxacin and ampicillin enhances glucose tolerance in mice. FASEB journal: official publication of the Federation of American Societies for Experimental Biology 22, 2416-2426.
Molofsky, A. B., Nussbaum, J. C., Liang, H. E., Van Dyken, S. J., Cheng, L. E., Mohapatra, A., Chawla, A., and Locksley, R. M. (2013). Innate lymphoid type 2 cells sustain visceral adipose tissue eosinophils and alternatively activated macrophages. The Journal of experimental medicine.
Nishimura, S., Manabe, I., Nagasaki, M., Eto, K., Yamashita, H., Ohsugi, M., Otsu, M., Hara, K., Ueki, K., Sugiura, S., et al. (2009). CD8+ effector T cells contribute to macrophage recruitment and adipose tissue inflammation in obesity. Nature medicine 15, 914-920.
Odegaard, J. I., and Chawla, A. (2013). The immune system as a sensor of the metabolic state. Immunity 38, 644-654.
Pastorelli, L., De Salvo, C., Mercado, J. R., Vecchi, M., and Pizarro, T. T. (2013). Central role of the gut epithelial barrier in the pathogenesis of chronic intestinal inflammation: lessons learned from animal models and human genetics. Frontiers in immunology 4, 280.
Petnicki-Ocwieja, T., Hrncir, T., Liu, Y. J., Biswas, A., Hudcovic, T., Tlaskalova-Hogenova, H., and Kobayashi, K. S. (2009). Nod2 is required for the regulation of commensal microbiota in the intestine. Proceedings of the National Academy of Sciences of the United States of America 106, 15813-15818.
Price, M. N., Dehal, P. S., and Arkin, A. P. (2009). FastTree: computing large minimum evolution trees with profiles instead of a distance matrix. Mol. Biol. Evol. 26, 1641-1650.
Reagan-Shaw, S., Nihal, M., and Ahmad, N. (2008). Dose translation from animal to human studies revisited. FASEB journal: official publication of the Federation of American Societies for Experimental Biology 22, 659-661.
Revelo, X. S., Tsai, S., Lei, H., Luck, H., Ghazarian, M., Tsui, H., Shi, S. Y., Schroer, S., Luk, C., Lin, G. H., et al. (2014). Perforin is a Novel Immune Regulator of Obesity Related Insulin Resistance. Diabetes.
Rousseaux, C., Lefebvre, B., Dubuquoy, L., Lefebvre, P., Romano, O., Auwerx, J., Metzger, D., Wahli, W., Desvergne, B., Naccari, G. C., et al. (2005). Intestinal antiinflammatory effect of 5-aminosalicylic acid is dependent on peroxisome proliferator-activated receptor-gamma. The Journal of experimental medicine 201, 1205-1215.
Sartor, R. B. (2010). Genetics and environmental interactions shape the intestinal microbiome to promote inflammatory bowel disease versus mucosal homeostasis. Gastroenterology 139, 1816-1819.
Teixeira, L. G., Leonel, A. J., Aguilar, E. C., Batista, N. V., Alves, A. C., Coimbra, C. C., Ferreira, A. V., de Faria, A. M., Cara, D. C., and Alvarez Leite, J. I. (2011). The combination of high-fat diet-induced obesity and chronic ulcerative colitis reciprocally exacerbates adipose tissue and colon inflammation. Lipids in health and disease 10, 204.
Turnbaugh, P. J., Ley, R. E., Mahowald, M. A., Magrini, V., Mardis, E. R., and Gordon, J. I. (2006). An obesity-associated gut microbiome with increased capacity for energy harvest. Nature 444, 1027-1031.
Upadhyay, V., Poroyko, V., Kim, T. J., Devkota, S., Fu, S., Liu, D., Tumanov, A. V., Koroleva, E. P., Deng, L., Nagler, C., et al. (2012). Lymphotoxin regulates commensal responses to enable diet-induced obesity. Nature immunology 13, 947-953.
Vazquez-Baeza, Y., Pirrung, M., Gonzalez, A., Knight, R. 2013. EMPeror: a tool for visualizing high-throughput microbial community data. GigaScience 2, 16.
Wagner, N., Lohler, J., Kunkel, E. J., Ley, K., Leung, E., Krissansen, G., Rajewsky, K., and Muller, W. (1996). Critical role for beta7 integrins in formation of the gut-associated lymphoid tissue. Nature 382, 366-370.
Wang, N., Wang, H., Yao, H., Wei, Q., Mao, X. M., Jiang, T., Xiang, J., and Dila, N. (2013). Expression and activity of the TLR4/NF-kappaB signaling pathway in mouse intestine following administration of a short-term high-fat diet. Experimental and therapeutic medicine 6, 635-640.
Wang, Q., Garrity, G. M., Tiedje, J. M., and Cole, J. R. (2007). Naive Bayesian classifier for rapid assignment of rRNA sequences into the new bacterial taxonomy. Applied and Environmental Microbiology 73, 5261-5267.
Wang, X., Ota, N., Manzanillo, P., Kates, L., Zavala-Solorio, J., Eidenschenk, C., Zhang, J., Lesch, J., Lee, W. P., Ross, J., et al. (2014). Interleukin-22 alleviates metabolic disorders and restores mucosal immunity in diabetes. Nature.
Wang, Y., Li, J., Tang, L., Charnigo, R., de Villiers, W., and Eckhardt, E. (2010). T-lymphocyte responses to intestinally absorbed antigens can contribute to adipose tissue inflammation and glucose intolerance during high fat feeding. PloS one 5, e13951.
Winer, D. A., Winer, S., Shen, L., Wadia, P. P., Yantha, J., Paltser, G., Tsui, H., Wu, P., Davidson, M. G., Alonso, M. N., et al. (2011). B cells promote insulin resistance through modulation of T cells and production of pathogenic IgG antibodies. Nature medicine 17, 610-617.
Winer, S., Chan, Y., Paltser, G., Truong, D., Tsui, H., Bahrami, J., Dorfman, R., Wang, Y., Zielenski, J., Mastronardi, F., et al. (2009a). Normalization of obesity-associated insulin resistance through immunotherapy. Nature medicine 15, 921-929.
Winer, S., Paltser, G., Chan, Y., Tsui, H., Engleman, E., Winer, D., and Dosch, H. M. (2009b). Obesity predisposes to Th17 bias. European journal of immunology 39, 2629-2635.
Wu, D., Molofsky, A. B., Liang, H. E., Ricardo-Gonzalez, R. R., Jouihan, H. A., Bando, J. K., Chawla, A., and Locksley, R. M. (2011). Eosinophils sustain adipose alternatively activated macrophages associated with glucose homeostasis. Science 332, 243-247.

1.-17. (canceled)

18. A method of treating high blood glucose comprising:
(a) selecting a patient having high blood glucose levels, and
(b) administering to said patient a gut anti-inflammatory agent.

19. The method of claim 18, wherein the high blood glucose is as a result of insulin resistance.

20. The method of claim 18, wherein the high blood glucose is as a result of glucose intolerance.

21. The method of claim 18, wherein the high blood glucose is a result of type 2 diabetes or obesity.

22. The method of claim 19, wherein the insulin resistance is a result of obesity.

23. The method of claim 20, wherein the glucose intolerance is as a result of type 1 diabetes, type 2 diabetes or obesity.

24. The method of claim 18, wherein the patient is selected on the basis of demonstrating insulin resistance.

25. The method of claim 18, wherein the patient is selected on the basis of demonstrating glucose intolerance.

26. The method of claim 24, wherein the insulin resistance demonstrated is as a result of the patient being obese.

27. The method of claim 25, wherein the glucose intolerance demonstrated is as a result of the patient having type 1 diabetes, type 2 diabetes or being obese.

28. The method of claim 18, wherein the patient is selected as having high blood glucose on the basis of the results of a fasting plasma glucose test, an oral glucose tolerance test, a random plasma glucose estimate, or an A1C test.

29. The method of claim 18, wherein the gut anti-inflammatory agent is a PPAR gamma analogue or a pharmaceutically acceptable salt, solvate, or ester of the PPAR gamma analogue.

30. The method of claim 29, wherein the PPAR gamma analogue is balsalazide or GED-0507-34.

31. The method of claim 18, wherein the gut anti-inflammatory agent is mesalamine (5-aminosalisylic acid, 5-ASA) or a derivative, analogue, prodrug or a pharmaceutically acceptable salt, solvate, or ester of any of the foregoing.

32. The method of claim 18, wherein the gut anti-inflammatory agent is mesalamine, sulfasalazine, asacol, delzicol, pentasa, lialda, apriso, olsalazine, balsalazide, or GED-0507-34, or a pharmaceutically acceptable salt, solvate, or ester of any of the foregoing.

33. The method of claim 18, wherein the route of administration is one of orally, intravenously, intraperitoneally, and rectally.

34. A method of treating high blood glucose comprising:
(a) selecting a patient having obesity, type 1 diabetes or type 2 diabetes, and
(b) administering to said patient a gut anti-inflammatory agent.

35. The method of claim 34, wherein the gut anti-inflammatory agent is a PPAR gamma analogue or a pharmaceutically acceptable salt, solvate, or ester of the PPAR gamma analogue.

36. The method of claim 35, wherein the PPAR gamma analogue is balsalazide or GED-0507-34.

37. The method of claim 34, wherein the gut anti-inflammatory agent is mesalamine (5-aminosalisylic acid, 5-ASA) or a derivative, analogue, prodrug or a pharmaceutically acceptable salt, solvate, or ester of any of the foregoing.

38. The method of claim 34, wherein the gut anti-inflammatory agent is mesalamine, sulfasalazine, asacol, delzicol, pentasa, lialda, apriso, olsalazine, balsalazide, or GED-0507-34, or a pharmaceutically acceptable salt, solvate, or ester of any of the foregoing.

39. The method of claim 34, wherein the route of administration is one of orally, intravenously, intraperitoneally, and rectally.


SEQUENCE LISTING (18 sequences)

SEQ ID NO: 1 - DNA, 21 bp, Artificial Sequence
  Primer sequence
agtccctgccctttgtacaca

SEQ ID NO: 2 - DNA, 19 bp, Artificial Sequence
  Primer sequence
cgatccgagggcctcacta

SEQ ID NO: 3 - DNA, 19 bp, Artificial Sequence
  Primer sequence
tcaccaccatggagaaggc

SEQ ID NO: 4 - DNA, 20 bp, Artificial Sequence
  Primer sequence
gctaagcagttggtggtgca

SEQ ID NO: 5 - DNA, 20 bp, Artificial Sequence
  Primer sequence
gacgacaggaaggtgaagag

SEQ ID NO: 6 - DNA, 20 bp, Artificial Sequence
  Primer sequence
acattccaccaccagcttgt

SEQ ID NO: 7 - DNA, 21 bp, Artificial Sequence
  Primer sequence
aagaacagcaacgagtaccgg

SEQ ID NO: 8 - DNA, 21 bp, Artificial Sequence
  Primer sequence
cattgtcactggtcagctcca

SEQ ID NO: 9 - DNA, 20 bp, Artificial Sequence
  Primer sequence
gatcaaagaggagccagtgc

SEQ ID NO: 10 - DNA, 20 bp, Artificial Sequence
  Primer sequence
tagatggtggctgctgagtg

SEQ ID NO: 11 - DNA, 20 bp, Artificial Sequence
  Primer sequence
gccctttggtgactttatgg

SEQ ID NO: 12 - DNA, 20 bp, Artificial Sequence
  Primer sequence
cagcaggttgtcttggatgt

SEQ ID NO: 13 - DNA, 20 bp, Artificial Sequence
  Primer sequence
tctgtcccggatctaccttg

SEQ ID NO: 14 - DNA, 20 bp, Artificial Sequence
  Primer sequence
gtagaatccaagcgcgaaac

SEQ ID NO: 15 - DNA, 20 bp, Artificial Sequence
  Primer sequence
gtgaggaagttcgtggaagg

SEQ ID NO: 16 - DNA, 20 bp, Artificial Sequence
  Primer sequence
tctgctcttgggtgatgatg

SEQ ID NO: 17 - DNA, 19 bp, Artificial Sequence
  Primer sequence
gccgctaagagcacagcaa

SEQ ID NO: 18 - DNA, 21 bp, Artificial Sequence
  Primer sequence
tccccactctgaaaatgagga